(19) United States
(12) Patent Application Publication
(10) Pub. No.: US 2026/0226398 A1
(43) Pub. Date: Aug. 6, 2026

(54) ALGAE BIOMASS

(71) Applicant: Algenuity BV, Rotterdam (NL)

(72) Inventors: Andrew Pearce Spicer, Stewartby (GB); Alexander Findlay Pudney, Stewartby (GB); Gino Schiano Schiano Di Visconte, Stewartby (GB); Emma Ransom-Jones, Stewartby (GB); Richard Thomas Smith, Stewartby (GB)

(73) Assignee: Algenuity BV, Rotterdam (NL)

(21) Appl. No.: 19/151,950

(22) PCT Filed: Jan. 29, 2024
(86) PCT No.: PCT/GB2024/050223

§ 371 Date: Jul. 29, 2025

(30) Foreign Application Priority Data
Jan. 30, 2023 (EP) 23154077.4
Jan. 30, 2023 (EP) 23154079.0

(51) Int. Cl. C12N 1/12; C07K 14/405; C12N 9/00; C12R 1/89

(52) U.S. Cl. CPC C12N 1/12; C07K 14/405; C12N 9/93; C12R 2001/89; C12Y 606/01001

(57) ABSTRACT

The invention relates to an algae biomass having particular values within a defined colour space. The invention further relates to a strain of Chlorella microalgae having a genetic mutation in genes encoding for specific subunits of magnesium chelatase, preferably magnesium chelatase subunit, and also relates to a method of producing a strain of Chlorella microalgae having such mutations.

### FIELD OF THE INVENTION

This invention relates to an algae biomass having particular values within a defined colour space. The invention further relates to a strain of Chlorella microalgae having a genetic mutation in magnesium chelatase subunit ChII. This invention also relates to a method of producing a strain of Chlorella microalgae having a genetic mutation in magnesium chelatase subunit ChII. This invention also relates to a composition comprising an algae biomass derived from the strain of Chlorella microalgae having a genetic mutation in magnesium chelatase subunit ChII and to its use as food ingredients amongst other applications.

### BACKGROUND

The widespread, ongoing adoption and resulting market growth of plant-based or flexitarian diets, as a result of increasing consumer awareness of the environmental and health consequences of a diet rich in animal protein, is often referred to as a “disruptive shift in consumption habits”. The retail plant-based protein market has achieved a global double-digit growth (14% CAGR) of worth 18.5 billion USD in 2020-21 and expected to reach >40 billion USD by 2026 with meat alternatives, plant-based milk and dairy and egg replacements representing >80% share that have a broad demographic appeal, including those who do not necessarily identify as vegetarian or vegan. Soy and pea protein (concentrates or isolates) currently dominate the global market for plant-based protein ingredients despite challenges around environmental impact, allergenicity and poor taste associated therewith. Therefore, food manufacturers are actively seeking alternative options; recognising the competitive advantage of plant-based proteins, such as oats, potato and chickpea, in addition to mycoprotein, insect protein and cultured or synthetic proteins, which have improved taste, nutrition, sustainability or allergenicity, consistent with evolving consumer preference. However, there is a growing urgent need for new sources of plant-based protein, beyond soy and pea, to meet consumer demand and expectations. First and foremost, these ingredients should deliver high nutritive value at an attractive cost and low environmental impact, while being readily scalable. Ingredients that can also provide functional, bioavailable protein with neutral taste and colour are even more desirable to the food and beverage manufacturer. Lastly, ingredients that can deliver on all of the above while delivering added health benefits to the consumer and potentially be produced without reliance on specific geographies, fitting strongly within a distributed robust food system have further value as we appreciate a world where external factors including global pandemics and conflicts can radically disrupt global supply chains and population health. All of these attributes align strongly to global priorities, for instance, within the UN's Sustainable Development Goals.

In this regard, algae in general have been identified as potential sources of vegetarian and/or vegan foods. While microalgae in particular, such as Chlorella sp. have been traditionally used as a food source for both human and animal consumption, recent trends in nutraceuticals and food industries have identified microalgae as a potential source of essential nutrients that provide several other benefits. For at least these reasons, Chlorella microalgae has a growing market opportunity as a food ingredient, largely owing to its high protein & fibre content and economical, heterotrophic production method.

For example, the green microalga, Chlorella vulgaris, has been produced commercially as a food and dietary supplement for at least the last 50 years. Moreover, Chlorella vulgaris is exempted from EU Novel Food Regulation (EU) 2015/2283-being as it was “on the market as a food or food ingredient and consumed to a significant degree (within the EU) before 15 May 1997”. In addition to being safe to eat for both humans and animals, both as a whole food and as an ingredient, Chlorella vulgaris is also present on the CIRS China List of approved cosmetic ingredients both as whole cell and as extract, as well as being included on the European Cosmetics Ingredients list.

Besides Chlorella vulgaris, other species of Chlorella such as Chlorella sorokiniana, as well as other microalgae related to the Chlorella genus especially those selected from the family Chlorellaceae, may be exploited commercially for various applications for example in food, nutraceuticals, cosmetics, and so on. For example, it will be appreciated that Chlorella sorokiniana UTEX 1230 and its equivalent strains (SAG 211-8k and CCAP 211/8k) represented in other culture collections, has an established history of consumption within the EU (and globally) before 15 May 1997, meaning that it does not fall under the scope of Regulation (EU) 2015/2283 of the European Parliament and of the Council of 25 Nov. 2015 on novel foods. (as clarified by the Czech Republic, Ministry of Agriculture in their Consultation of 4 Mar. 2022). Chlorella sorokiniana will be likely to emerge as a preferred species for food applications as the market for microalgae-based plant protein continues to grow, owing to its productive growth rate when cultivated heterotrophically on glucose, favourable regulatory status in major markets and higher protein content.

Despite the advantages offered, the use of Chlorella microalgae has been limited at least in part for certain market applications, including acceptance as a conventional food source and widespread use as a cosmetics and/or a personal care ingredient. The limited use of Chlorella microalgae is largely due to the dark-green colour, along with undesirable aroma and flavour that are often associated with the normal levels of chlorophyll in the wild-type Chlorella microalgae; for example, chlorophyll usually comprises between 1-2% of the dry cell weight of Chlorella vulgaris.

In order to overcome these problems, to promote their use in food or as food ingredients, Chlorella microalgae biomass is either used for specific products and markets where acceptance would be more expected in spite of the less-appealing colour, appearance and/or taste and smell, or used at very low incorporation rate, or often mixed with other components (food or food ingredients) with a different colour, stronger aroma and/or flavour or omitted from certain products/markets altogether. However, the latter techniques may still fail to overcome the undesirable colour, aroma and/or flavour associated with Chlorella microalgae. Consequently, these microalgae do not have the most desirable properties to be used as food, cosmetic and/or personal care ingredients.

There exists, therefore, a need to overcome the aforementioned drawbacks associated with algae biomass and their use for human consumption and other market needs.

It is the object of the invention to provide an algae biomass having improved properties, in particular properties related to colour, to existing materials of this type for their use in consumer products. It is a further object of the invention to provide a genetically stable, non-recombinant, variant strain of Chlorella microalgae from which this algae biomass may be derived. Beneficially, since the improved colour of the biomass corresponds to a low chlorophyll content, the biomass of the invention also exhibits improved organoleptic properties relating to taste and smell.

### SUMMARY OF THE INVENTION

A first aspect of the invention provides an algae biomass, wherein said biomass has a L* value in an L*a*b* colour space of greater than about 78.

The term “CIEXYZ” (CIE 1931 colour space) is used to refer to an early reference colour space adopted by the International Commission on Illumination (Commission Internationale de l'Eclairage, abbreviated to CIE) in 1931 after experimentation of human perception of colour. The colour space was made to model the average human's sensitivity to different colours under a specific light source (standard illuminant) and angle of illumination (standard observer). The colour space is produced from three tristimulus values, X, Y and Z. Y is the luminance (or brightness), Z roughly equates to blue, and X is a mixture of red, green and blue.

The term “CIELAB” (or “L*a*b*”) refers to a colour space that was adopted by the CIE in 1976, to produce a more perceptually uniform space compared to CIEXYZ, where the values are coordinated with a colour. The CIELAB values cover the entire range of human colour perception as three values, L* indicating lightness (0=black, 100=white), a* (negative values=green, positive values=red) and b* (negative values=blue, positive values=yellow). The L* coordinate nominally ranges from 0 to 100. The range of a* and b* coordinates is technically unbounded, though it is commonly clamped to the range of −128 to 127. CIELAB is calculated from the older CIEXYZ values. The formula for converting the CIEXYZ values to CIELAB is:

$${L}^{*} = 116 f ( \frac{Y}{{Y}_{n}} ) - 16$$

$${a}^{*} = 500 ( f ( \frac{X}{{X}_{n}} ) - f ( \frac{Y}{{Y}_{n}} ) )$$

$${b}^{*} = 200 ( f ( \frac{Y}{{Y}_{n}} ) - f ( \frac{Z}{{Z}_{n}} ) )$$

$$where$$

$$f ( I ) = {I}^{1 / 3}$$

X, Y, Z describe the colour stimulus (CIEXYZ) measured, whilst Xn, Yn and Zn describe a specified white achromatic reference illuminant (light source). I, refers to the reflectance value or the ratios of Y/Yn, X/Xn, or Z/Zn. If I is <0.008856 (very dark colours), a different coefficient is used for f; as reviewed by Luo et al. (Luo et al. 2001; https://doi.org/10.1002/col. 1049). CIE recommends the use of CIE Standard illuminant D65, (which corresponds to the average midday light in the Western hemisphere).

For the CIE 1931 (2°) standard colorimetric observer and assuming normalization where reference white=Y=100, the values are:

For Standard Illuminant D65:
Xn=95.0489,
Yn=100,
Zn=108.8840

Alternatively, using the updated recommended alternative (CIE1964) 10° standard colorimetric observer, and assuming normalization where reference white=Y=100, the values are:

For Standard Illuminant D65:
Xn=94.8110,
Yn=100,
Zn=107.304

There are a number of alternative standard illuminants (for example but not exclusively, Illuminant A, B and C, E, F) and standard observers (for example, but not exclusively 2° and) 10° that can be used to calculate CIELAB. These can be converted interchangeably, but should be stated.

A single unit value, ΔE*ab (or CIE76), can be calculated; being a measure of change in visual perception of two given colours within the CIELAB colour space. The values are on a scale of 0-100, where values below 1 are not perceptible to the human eye, 1-2 is perceptible through close observation, 2-10 perceptible at a glance, 11-49 colours more similar than the opposite, 100 colours are exactly the same. The formula for ΔE*ab is below:

$$Δ {E}_{a b}^{*} = \sqrt{{( {L}_{2}^{*} - {L}_{1}^{*} )}^{2} +} {( {a}_{2}^{*} - {a}_{1}^{*} )}^{2} + {( {b}_{2}^{*} - {b}_{1}^{*} )}^{2}$$

The term “Hunter L, a, b” is used to refer to a colour space that can be used instead of CIELAB. The Hunter L, a, b scale is very similar to CIELAB, but uses a square root, rather than cubed root, transformation of the CIEXYZ values.

L is a correlate of lightness and is calculated with the following formula:

$$L = 100 \sqrt{\frac{Y}{{Y}_{n}}}$$

Where Yₙ is the Y tristimulus value of a specified white object. The L value will be between 0 (black) and 100 (white). a and b are opponent colour axes with a representing redness (positive) versus greenness (negative). a is calculated by this formula:

$$a = {K}_{a} ( \frac{\frac{X}{{X}_{n}} - \frac{Y}{{Y}_{n}}}{\sqrt{\frac{Y}{{Y}_{n}}}} )$$

Kₐ is a coefficient that depends upon the illuminant (for D65, Ka is 172.30) and Xn is the X tristimulus value of the specified white object. B is positive for yellow colours and negative for blue colours and is calculated by the following formula:

$$b = {K}_{b} ( \frac{\frac{Y}{{Y}_{n}} - \frac{Z}{{Z}_{n}}}{\sqrt{\frac{Y}{{Y}_{n}}}} )$$

Kb is a coefficient that depends upon the illuminant (for D65 Kb is 67.20).

The CIEXYZ values can also be used to calculate a single index value, WI. WI is an index of how closely a colour matches the properties of a perfect reflecting diffuser. There are various WI index calculations that have different applications and bias on different colours (Gantz index, Berger index, WI (Hunter), WI (Stensby), Taube Index. Judd Index, MacAdam Index, CIE WI). The CIE recommended WI is published in ASTM Method E313 (DOI: 10.1520/E0313-20) and recommended only for relative evaluation using the same instrument at a given time.

The present invention is an algae biomass that has an L* value, in terms of CIELAB L*a*b* colour space values, that is higher than that of other known algae biomass materials. This is particularly advantageous as such an algae biomass therefore has improved properties that relate to consumer acceptance of consumer goods (e.g. food products) that contain the algae biomass, and in particular improved visual properties, improved taste properties, and improved smell properties.

As described above, the L* range in the CIELAB colour space has a range of 0 to 100. The algae biomass of the present invention has an L* value of greater than about 78. Preferably, the algae biomass has an L* value of greater than about 79, about 80, about 81, about 82, about 83, about 84, or about 85. Particularly preferably, the algae biomass has an L* value of greater than about 81.0, about 81.5, about 82.0, about 82.5, about 83.0, about 83.5, about 84.0, or about 84.5.

The algae biomass may have an L* value in the range of from between 78 and 90. Preferably, the algae biomass has an L* value in the range of from 79 to 85. Particularly preferably, the algae biomass has an L* value in the range of from 81 to 85.

It has, surprisingly, been found that L* values of above about 81, such as in the range of from between 81 and 85, correspond to algae biomass that has significantly improved properties, particularly visual properties and/or organoleptic properties (i.e. properties relating to smell and taste) that are relevant to consumer acceptance when the algae biomass is incorporated into consumer goods, such as food products. This particularly beneficial value, or range, of L* values, has not previously been described in the art in relation to algae biomass of this type.

The algae biomass preferably has an a* value in an L*a*b* colour space in the range of between 0.1 and 4.0. Particularly preferably, the algae biomass has an a* value in the range of between 1.5 and 3.5. Still more preferably, the algae biomass has an a* value in the range of between 1.8 and 3.1.

The algae biomass preferably has a b* value in an L*a*b* colour space in the range of between 10 and 27. Particularly preferably, the algae biomass has a b* value in the range of between 15 and 25. Still more preferably, the algae biomass has a b* value in the range of between 15 and 20, such as between 16 and 19.

In one preferred embodiment, the algae biomass has a L* value in an L*a*b* colour space in the range of between 81.0 and 85.0; an a value of in the range of between 1.8 and 3.1; and a b* value in the range of between 16.0 and 19.0.

In one preferred embodiment, the algae biomass has a L* value in an L*a*b* colour space in the range of between 82.0 and 89.0; an a value of in the range of between 0.4 and 2.2; and a b* value in the range of between 13.0 and 18.0.

In one preferred embodiment, the algae biomass has a L* value in an L*a*b* colour space in the range of between 80.0 and 87.0; an a value of in the range of between 0.7 and 2.0; and a b* value in the range of between 15.0 and 19.0.

In one preferred embodiment, the algae biomass has a L* value in an L*a*b* colour space in the range of between 85.0 and 90.0; an a* value of in the range of between 0.3 and 1.2; and a b* value in the range of between 9.0 and 13.0.

In one preferred embodiment, the algae biomass has a L* value in an L*a*b* colour space in the range of between 87.0 and 89.0; an a* value of in the range of between 0.5 and 0.6; and a b* value in the range of between 11.0 and 12.0.

Preferably, the algae biomass has a ratio of the b* value to the a* value of at least 5:1, preferably of at least 10:1, more preferably of at least 20:1, still more preferably of at least 30:1.

Preferably, the algae biomass has a ratio of the b* value to the a* value of less than 40:1, preferably of less than 30:1, more preferably of less than 20:1, still more preferably of less than 10:1.

It has surprisingly been found that L*a*b* values in this combination of ranges correspond to algae biomass that has significantly improved visual properties, particularly visual properties that are relevant to consumer acceptance when the algae biomass is incorporated into consumer goods, such as food products. This particularly beneficial value, or range, of L* values, has not previously been described in the art in relation to algae biomass of this type.

The algae biomass as described herein preferably maintains a minimum protein content of at least 20%, 25%, 30%, 35%, 40%, or 45% w/w. For example, the protein content of the algae biomass may be from 20%, 25%, 30%, 35%, 40% or 45% w/w up to 30%, 35%, 40%, or 45% w/w.

In an embodiment, the algae biomass has a protein content in a range of 50-85% w/w. For example, the protein content of the algae biomass may be 50, 55, 60, 65, 70, 75 or 80% w/w up to 55, 60, 65, 70, 75, 80 or 85% w/w. Optionally, the algae biomass has a protein content in a range of 50-75% w/w, preferably 50-70% w/w, more preferably 50-60% w/w. For example, the protein content of the algae biomass may be 50, 55, 60, 65 or 70% w/w up to 55, 60, 65, 70 or 75% w/w, preferably 50, 55, 60 or 65% w/w up to 55, 60, 65 or 70% w/w.

The algae biomass as described herein may be in the form of a powder or flour, e.g. an algal flour.

The term “algal flour” (used interchangeably herein with the term “algae flour” or “microalgae flour”) is used to refer to an edible composition comprising a plurality of particles of algae biomass. Optionally, the plurality of particles of algae biomass is any one of: whole cells, lysed cells or a mixture thereof. More optionally, the algal flour comprises one or more of significant digestible proteins, dietary fibre content, associated water binding attributes, healthy oil delivering attributes, spices, herbs, a flow agent, an antioxidant and so forth. It may be appreciated that the algal flour lacks visible oil and is preferably in a powdered form. The algal flour can be produced under current Good Manufacturing Practice (cGMP) conditions using any method known in the art.

A second aspect of the invention provides a Chlorella microalgae having a first mutation in at least one gene that encodes for phytoene desaturase or a subunit thereof, and wherein said Chlorella microalgae has a second mutation in at least one gene that encodes for magnesium chelatase or a subunit thereof.

It is understood that the invention, methods and approach described herein also apply to the identification and isolation of variant strains of other algal biomass (such as green algae). The term “algal biomass” (or “algae biomass”) as used herein refers to a biomass derived from algae, such as Chlorella microalgae. It will be appreciated that the algal biomass may typically be selected from the Chlorellaceae taxonomic family of green algae of which notable genera include the true Chlorella species, such as but not limited to Chlorella sorokiniana or Chlorella vulgaris in addition to other species that have historically been identified, consumed or commercially sold as genus Chlorella; including, but not limited to Parachlorella kessleri, Auxenochlorella protothecoides, Auxenochlorella pyrenoidosa, or Heterochlorella luteoviridis.

Algae produce a number of pigments including chlorophylls and carotenoids, which capture energy from light as part of the process of photosynthesis. These pigments are critical for photosynthesis and, as such, their production is tightly controlled by a number of enzymes as part of either the chlorophyll or carotenoid synthesis pathways.

Accordingly, “magnesium chelatase” (EC 6.6.1.1) is an enzyme that catalyses the first committed step of the chlorophyll synthesis pathway; being the insertion of Mg²⁺ into protoporphyrin IX. Magnesium chelatase is a highly-conserved enzyme composed of three subunits: ChII, ChID, and ChIH. The subunits are postulated to have distinct roles in forming the catalytically-active holoenzyme that, ultimately, performs the magnesium chelation reaction; broadly, ChII and ChID are thought to form an ATP-associated complex, while ChIH binds to the magnesium ion, leading to the formation of the active Mg chelatase holoenzyme (Xhang et al. 2018; DOI: 10.3389/fpls.2018.00720).

Phytoene desaturase (EC 1.3.5.5) is an enzyme essential to the carotenoid biosynthesis pathway and controls the conversion phytoene into lycopene. Within the carotenoid synthesis pathway, “phytoene desaturase” (EC: 1.3.5.5) converts phytoene into zeta-carotene, which forms the basis of all other plant carotenoids.

Geranylgeranyl diphosphate synthase (EC 2.5.1.29) is an enzyme required for the synthesis of Geranylgeranyl diphosphate (GGPP), which is the precursor for the biosynthesis of carotenoids and chlorophylls.

It has surprisingly been found that Chlorella microalgae that have a first mutation in a gene that encodes for phytoene desaturase, or a subunit thereof, and additionally have a second mutation in a gene that encodes for magnesium chelatase, or a subunit thereof, have improved properties relating to the use of such microalgae and algae biomass derivatives thereof in consumer goods (e.g. food products). In particular, this combination of mutations has surprisingly been found to result in Chlorella microalgae from which algal biomass may be derived that has an L* value, in terms of CIELAB L*a*b* colour space values, that is higher than that of other known algae biomass materials, as described above. It has also surprisingly been found that this combination of mutations results in Chlorella microalgae from which algal biomass may be derived that has L*a*b* values, in terms of CIELAB L*a*b* colour space values, that is particularly beneficial.

In preferred Chlorella microalgae as hereinbefore described, the second mutation is in a gene that encodes for subunit ChII of magnesium chelatase or subunit ChIH of magnesium chelatase.

In particularly preferred Chlorella microalgae as hereinbefore described wherein the second mutation is in a gene that encodes for subunit ChII of magnesium chelatase.

In some preferred Chlorella microalgae as hereinbefore described, a mutation is present in a gene that encodes for Geranylgeranyl diphosphate synthase (GGPP), or a subunit thereof. Thus, some preferred Chlorella microalgae of the invention have a first mutation in a gene that encodes for phytoene desaturase, or a subunit thereof, and additionally have a second mutation in a gene that encodes for magnesium chelatase, or a subunit thereof, and additionally have a third mutation in a gene that encodes for Geranylgeranyl diphosphate synthase, or a subunit thereof.

It has surprisingly been found that Chlorella microalgae having a first mutation in a gene that encodes for phytoene desaturase, or a subunit thereof, and additionally having a second mutation in a gene that encodes for subunit ChII of magnesium chelatase have particularly improved properties relating to the use of such microalgae and algae biomass derivatives thereof in consumer goods (e.g. food products). In particular, this combination of mutations has surprisingly been found to result in Chlorella microalgae from which algal biomass may be derived that has an L* value, in terms of CIELAB L*a*b* colour space values, that is higher than that of other known algae biomass materials, as described above. It has also surprisingly been found that this combination of mutations results in Chlorella microalgae from which algal biomass may be derived that has L*a*b* values, in terms of CIELAB L*a*b* colour space values, that is highly beneficial.

It has surprisingly been found that Chlorella microalgae that have a first mutation in a gene that encodes for phytoene desaturase, or a subunit thereof, and additionally have a second mutation in a gene that encodes for magnesium chelatase, or a subunit thereof, and additionally have a third mutation in a gene that encodes for Geranylgeranyl diphosphate synthase, or a subunit thereof, have even further improved properties relating to the use of such microalgae and algae biomass derivatives thereof in consumer goods (e.g. food products). In particular, this combination of mutations has surprisingly been found to result in Chlorella microalgae from which algal biomass may be derived that has an L* value, in terms of CIELAB L*a*b* colour space values, that is higher than that of other known algae biomass materials, as described above. It has also surprisingly been found that this combination of mutations results in Chlorella microalgae from which algal biomass may be derived that has L*a*b* values, in terms of CIELAB L*a*b* colour space values, that is highly beneficial.

Preferably, in the Chlorella microalgae as hereinbefore described, the second mutation results in a loss of function, but could also result in a reduction in function. This is more likely to be caused by a frameshift mutation as a result of an INDEL, but could also be caused by a SNP resulting in a nonsynonymous mutation with either the protein rendered nonfunctional due to a change in the amino acid structure, or in a premature stop codon resulting in a truncated protein.

The present invention overcomes the drawbacks mentioned above by using a whole algal cell, selected from Chlorella microalgae having a reduced chlorophyll content less than 0.5 mg/g dry cell weight. The algae strain of the invention is produced by a non-recombinant method, and the invention is, therefore, a non-genetically modified whole algal cell having genetic stability as compared to its progenitor cells. In particular, the strain of the invention (or hereafter referred to as the “variant(s)” or “variant strain(s)”) of Chlorella microalgae enable improved organoleptic properties as well as a wide range of applications thereof, such as whole-cell algal ingredients and their applications.

It is understood that the invention, methods and approach described herein also apply to the identification and isolation of variant strains of other algal biomass (such as green algae) that are haploid genetically. The term “algal biomass” (or “algae biomass”) as used herein refers to a biomass derived from algae, such as Chlorella microalgae. It will be appreciated that the algal biomass may typically be selected from the Chlorellaceae taxonomic family of green algae of which notable genera include the true Chlorella species, such as but not limited to Chlorella sorokiniana or Chlorella vulgaris in addition to other species that have historically been identified, consumed or commercially sold as genus Chlorella; including, but not limited to Parachlorella kessleri, Auxenochlorella protothecoides, Auxenochlorella pyrenoidosa, or Heterochlorella luteoviridis.

The term “chlorophyll” as used herein refers to a group of green pigments contained in cells of green plants. Chlorophyll is essential for photosynthesis and allows photosynthetic organisms to absorb energy from sunlight (absorbing blue and red lights and reflecting green light from the visible region of the electromagnetic spectrum). It will be appreciated that the chlorophyll content is associated with at least one of: chlorophyll a (α-chlorophyll or Chl-a) and/or chlorophyll b (β-chlorophyll or Chl-b). Chlorophyll a is a primary photosynthetic pigment, which participates directly in the light-driven reactions of photosynthesis, while chlorophyll b is an accessory pigment operable to collect energy primarily from blue wavelengths of sunlight and pass it on to chlorophyll a. Moreover, the chlorophyll content can be influenced by cultivation conditions, in particular the absence or presence of light. In the dark, chlorophyll content can be reduced or even significantly suppressed.

Moreover, the protein content of the Chlorella microalgae may be identified as the protein concentrate therein. The term “protein concentrate” refers to a certain threshold level of protein content, typically produced using aqueous or mild alkali extraction (pH 7-10) of proteins and soluble carbohydrates. The insoluble residue, mostly carbohydrate, is thus removed by centrifugation, followed by precipitation of protein at its isoelectric point (pH~4.5). The precipitated protein is separated by mechanical decanting, washed, and neutralized to a pH of 6.8 and then spray-dried.

Moreover, a reduced chlorophyll content of the variant strain of Chlorella microalgae beneficially affects its organoleptic properties (for example, taste and smell).

It will be appreciated that the Chlorella microalgae of the invention are not capable of photoautotrophic growth, being that they have a chlorophyll content in a range of 0.001-0.5 mg/g dry cell weight. Without wishing to be bound by theory, it will be understood that this low chlorophyll content is a result of the mutations in genes which encode for phytoene desaturase (or a subunit thereof) and which encode for magnesium chelatase (or a subunit thereof), preferably subunit ChII or ChIH of magnesium chelatase and more preferably subunit ChII of magnesium chelatase as hereinbefore described.

In an embodiment, the strain of Chlorella microalgae as defined herein is a modified strain of a Chlorella microalgae species. Typically, the modified strain (namely, ‘variant strain’) of Chlorella microalgae is a modification of the progenitor cells of the Chlorella microalgae. Herein, the term “progenitor” refers to a wild-type or parental strain of the Chlorella microalgae.

The term “wild-type strain” (or wild-type) refers to a typical form of an organism as it occurs in nature. Specifically, the wild-type is a typical form of an organism of a species comprising a set of genes characteristic to a naturally existing organism of that species, i.e. comprising normal occurrence of a gene at a locus, and exhibiting the associated phenotypes thereof. The wild-type strain of Chlorella microalgae can be obtained from its usual dwelling sites such as land, rivers, ponds, lakes, brackish water, wastewater and the like. The naturally existing wild-type strain of Chlorella microalgae is able to grow autotrophically by performing photosynthesis (producing a biomass of alga by utilizing sunlight, carbon dioxide, water and a few nutrients). However, the wild-type strain of Chlorella microalgae can also be cultivated using heterotrophic and/or mixotrophic growth modes. Wild-type strains of Chlorella microalgae are haploid in their normal growth phase, i.e. have only one copy of the genome, thereby making Chlorella microalgae particularly amenable to a phenotypic trait improvement approach using genetics as, for some traits, a single genetic change could yield the desired phenotype. Furthermore, being haploid, these variant or improved strains are likely to be genetically stable as there is essentially no capacity of the mutant strain to easily correct or revert to the wild-type state; moreover, there is no other genetic copy of the DNA that can act as a correction template to facilitate this process. Here, comparative taxonomic analysis by alignment of ITS2 genetic sequences using ClustaW2 software (Madeira et al. 2019; DOI: 0.1093/nar/gkz268) was used to establish that proprietary Chlorella vulgaris strain 4TC3/16 (4TC3) is a wild-type strain of Chlorella vulgaris; being that is taxonomically identical to the culture collection type strain of Chlorella vulgaris 211/11b.

The term “parent strain” as used herein, refers to a progenitor organism that, during the process of division, replicates its DNA, which is then inherited by an offspring or daughter cell thereof. Specifically, Chlorella microalgae reproduce asexually by multiple fission, with the basic rule that one mother cell reproduces its DNA synchronously to produce at least two daughter cells per division event (or burst). Occasionally, a division burst may comprise four, eight and rarely, sixteen daughter cells (Mandalam and Palsson 1997; DOI 10.1023/A: 1018310008826). The number of Chlorella microalgae daughter cells produced per division burst is thought to be modifiable by environmental factors such as light and temperature-being as they directly affect growth rate, and consequently, the coordination between DNA replication and division events in the cell cycle (Bišová and Zachleder 2014; DOI: 10.1093/jxb/ert466). Given this asexual method of whole genome reproduction and inheritance, it can be understood that Chlorella microalgae strains exhibit an extremely high degree of genetic stability between generations. Further, in the context of a mutagenesis campaign, the parent strain may be a wild-type strain of Chlorella microalgae or a variation (i.e. a genetic variant) of the wild-type strain of Chlorella microalgae. The term “parental strain”, therefore, may also refer to a genetic variant or subtype of Chlorella microalgae, preferably a previous generation.

It will be appreciated that a variation of the wild-type strain of Chlorella microalgae differs from the parent strain (namely, the wild-type strain) only by the mutated gene(s) (and in some cases closely linked genes). Such variant strains of Chlorella microalgae are valuable in understanding the effect of a single or multiple gene mutations in the organism. The variation of the wild-type strain of Chlorella microalgae may be a genetic mutant.

In an embodiment, the Chlorella microalgae species is selected from Chlorella sorokiniana or Chlorella vulgaris. The term “Chlorella vulgaris” as used herein, refers to a species of single-cell aquatic plant, termed microalgae, falling under Division “Chlorophyta” within the plant taxonomic Kingdom. The full taxonomic assignment is: Biota Plantae (Kingdom) Viridiplantae (Subkingdom) Chlorophyta (Phylum (Division)) Chlorophytina (Subphylum (Subdivision)) Trebouxiophyceae (Class) Chlorellales (Order) Chlorellaceae (Family) Chlorella (Genus) Chlorella vulgaris (Species). The microalgae are photosynthetic organisms that grow in diverse habitats ranging from regions of varying hardness of growth medium (such as soil or water), humidity, salinity, light-access, and temperature conditions, such as land, rivers, ponds, lakes, sea, brackish water, wastewater and the like. Typically, the wild-type strains of Chlorella vulgaris are associated with a dark-green colour, a specific smell (such as aquatic, fish-like, earthy or mouldy smell), an unpleasant taste, in addition to a cell wall, which has glucosamine as its main component, and generally comprises an alkali soluble hemicellulose fraction, and a residual insoluble fraction, collectively forming the rigid wall.

The term “Chlorella sorokiniana” as used herein, refers to a species of green microalgae, that can grow in freshwater and consumes both organic and inorganic carbon, falling under Division “Chlorophyta” within the plant taxonomic Kingdom. The full taxonomic assignment is: Biota Plantae (Kingdom) Viridiplantae (Subkingdom) Chlorophyta (Phylum (Division)) Trebouxiophyceae (Class) Chlorellales (Order) Chlorellaceae (Family) Chlorella (Genus) Chlorella sorokiniana (Species). Typically, the wild-type strains of Chlorella sorokiniana are associated with a characteristic emerald-green colour and pleasant grass odour, in addition to a cell wall which has glucosamine as its main component, and generally comprises an alkali soluble hemicellulose fraction, and a residue fraction, the rigid wall. The hemicellulose fraction of the Chlorella sorokiniana cell wall may contain 50% higher proportion of rhamnose, compared to Chlorella vulgaris. In addition, Algenan (previously “sporopollenin”), a highly-resistant biopolymer, is a long-suspected component of the Chlorella sorokiniana UTEX1230 cell wall (e.g. Rosen et al. 1985, DOI: 10.1016/0168-9452 (85) 90061-5; Kodner et al. 2009, 10.1016/j.orggeochem.2009.05.003). Further, the highly-conserved N-terminal glycan structures of the cell walls within genus Chlorella are sufficiently diverse that they can be used to differentiate between species (Mócsai et al 2020; DOI: 10.1111/tpj.14718).

In addition to the composition of cell wall carbohydrates and general chemotaxonomy, Chlorella sorokiniana (such as type-strain UTEX 1230) may be further distinguished from Chlorella vulgaris on the basis that the former tolerates a higher cultivation temperature; reportedly up-to 39° C. for UTEX 1230 (Sorokin & Myers 1953; DOI: 10.1126/science.117.3039.330), whereas the latter typically exhibits an upper temperature tolerance of 28-30° C. (Kessler 1985; DOI: 10.1007.bf02418020).

In addition to being phototrophs, both Chlorella vulgaris and Chlorella sorokiniana exhibit the ability to grow heterotrophically, on glucose (or other suitable organic carbon source), in the absence of light. Further, both Chlorella vulgaris and Chlorella sorokiniana are cultivable in mixotrophic growth mode; using a mixture of light and glucose, or other suitable organic carbon source.

The wild-type or parent strain of Chlorella microalgae may be obtained from their natural habitats or from laboratory cultures. The obtained strains of Chlorella microalgae are genetically defined as Chlorella microalgae using PCR amplification, sequencing and alignment of the genetic material with a reference sequence. Examples of useful genetic sequencing targets for the purpose of taxonomic identification of Chlorella microalgae include, but are not limited to: 18S rRNA gene sequence, the internally transcribed spacer (ITS) regions between the 18S rRNA gene, 5.8S rRNA gene and the 28S rRNA gene sequence. Such regions have been used extensively for intra and inter genus phylogenetic analysis of the Chlorellaceae (green algae) family (Huss et al. 1999; DOI: 10.1046/j. 1529-8817.1999.3530587.x, Krienitz et al., 2015; DOI: 10.1016/j.tplants.2014.11.005, Darienko and Proschold 2015; DOI: 10.1111/jpy.12279, and Heeg and Wolf 2015; DOI: 10.1016/j.plgene.2015.08.001). Other statistics and additional sequences derived from whole genome sequencing are another method for strain identification.

Typically, Chlorella sorokiniana or Chlorella vulgaris are robust species with a high consumer interest owing to their biotechnological and economical potential including, but not limited to, a wide variety of primary biomolecules (such as proteins, carbohydrates and lipids) and several intermediate compounds, nutritional value, and so forth.

In an embodiment, the strain of Chlorella microalgae is obtained from a parent strain of Chlorella microalgae, by performing mutagenesis of the parent strain of Chlorella microalgae. The term “mutagenesis” as used herein, relates to a technique of inducing mutations by artificially exposing the organism to mutagens using laboratory procedures. Mutagens have the effect of increasing the frequency of genetic mutation over and above the natural frequency of spontaneously occurring mutations. The variation of the wild-type or parental strain may be a genetic mutant.

In an embodiment, mutagenesis is performed by exposure of the parent strain of Chlorella microalgae to a mutagenic chemical. It will be appreciated that chemical mutagenesis is not considered to produce Genetically Modified Organisms (GMOs) as defined by the current EU legislation; European Union Directive 2001/18/EC (Annex 1B).

In an embodiment, the mutagenic chemical is an alkylating agent. The term “alkylating agent” as used herein, refers to one or more classes of alkylating agents functioning as mutagens. Generally, the alkylating agents transfer alkyl groups (such as methyl or ethyl group) to macromolecules (such as bases, or the backbone phosphate groups of the nucleic acids) under physiological conditions. Typically, the alkyl group acts on nucleophilic sites of the macromolecule, for example, nitrogen or oxygen nucleophiles in DNA (as described by Gates 2009; DOI: 10.1021/tx900242k). Such transfers result in alkylation of bases (for example guanine) and subsequent mispairing of said base during DNA replication (with for example, thymine instead of cytosine). Normally, the alkylating agents that function as mutagens include but are not limited to: sulphur mustards, nitrogen mustards, epoxides, ethylene imines, alkyl alkanesulphonates, dialkyl sulphates, beta-lactones, diazo compounds and nitroso compounds. Examples of such alkylating agents from each of these respective classes include: mustard gas, nitrogen mustard (HN2), ethylene oxide (EO), diepoxybutane (DEB), ethyleneimine (EI), triethylenemelamine (TEM), ethyl methanesulphonate (EMS) and methyl methansulphonate (MMS), diethylsulphate (DES), beta-propiolactone, diazomethane, N-Nitroso-N-methylurea (NMU) and N-methyl-N′-nitro-N-nitrosoguanidine (NG or NTG or MNNG) (as described by Auerbach 1976; DOI: 10.1007/978-1-4899-3103-0_16).

In an embodiment, the concentration of the mutagenic chemical is in a range from 0.1 to 2.0 M. Typically, mutagenesis is performed by exposure of the parent strain of Chlorella microalgae to a sub-lethal quantity of the mutagenic chemical. The sub-lethal quantity of the mutagenic chemical is defined as the amount or quantity of the mutagenic chemical that results in less than 100% kill of the parent strain of Chlorella microalgae in a given time. The concentration of the mutagenic chemical may be 0.1 to 2.0 M, 0.2 to 2.0 M, 0.5 to 2.0 M, 0.7 to 1.0 M. The concentration of the mutagenic chemical may be for example from 0.1, 0.2, 0.3, 0.4, 0.5, 0.6, 0.7, 0.8, 0.9, 1.0, 1.1, 1.2, 1.3, 1.4, 1.5, 1.6, 1.7, 1.8 or 1.9 M up to 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 M, preferably 0.2, 0.3, 0.4, 0.5, 0.6, 0.7, 0.8, 0.9, 1.0, 1.1, 1.2, 1.3, 1.4, 1.5, 1.6, 1.7, 1.8 or 1.9 M up to 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 M, 0.5, 0.6, 0.7, 0.8, 0.9, 1.0, 1.1, 1.2, 1.3, 1.4, 1.5, 1.6, 1.7, 1.8 or 1.9 M up to 0.6, 0.7, 0.8, 0.9, 1.0, 1.1, 1.2, 1.3, 1.4, 1.5, 1.6, 1.7, 1.8, 1.9 or 2.0 M, 0.7, 0.8, 0.9, 1.0, 1.1, 1.2, 1.3, 1.4, 1.5, 1.6, 1.7, 1.8 or 1.9 M up to 0.8, 0.9, 1.0, 1.1, 1.2, 1.3, 1.4, 1.5, 1.6, 1.7, 1.8, 1.9 or 2.0 M, 0.1, 0.2, 0.3, 0.4, 0.5, 0.6, 0.7, 0.8 or 0.9 M up to 0.2, 0.3, 0.4, 0.5, 0.6, 0.7, 0.8, 0.9 or 1.0. In an example, the concentration of the mutagenic chemical is 0.2 M of EMS that is non-lethal to the Chlorella microalgae species. In another example, the sub-lethal quantity is 0.2 M of MMS that produces a 20% lethality to the Chlorella microalgae species (referred to as “mutagen kill” hereafter). In yet another example, the sub-lethal quantity is 0.8 M of EMS that produces a 40% mutagen kill. In still another example, the sub-lethal quantity is 0.8 M of MMS that produces a 60% mutagen kill.

In case of EMS, repeated replication of such mispaired DNA can result in a transition mutation, wherein original G: C base pairs change to A: T base pairs, thereby changing the genetic makeup of the organism. In such case, the replication of such mutated DNA may create heritable missense mutations or nonsense mutations within coding sequences or impacting gene expression or gene function by compromising regulatory sequence functionality including RNA splice-site mutations or promoter or other regulatory sequence mutations. Beneficially, the use of alkylating agents as mutagenic chemicals for plant breeding, for human consumption, is not considered to produce Genetically Modified Organism (GMOs) as defined by the current EU legislation; European Union Directive 2001/18/EC (Annex 1B), and is therefore acceptable for further applications in various industries, such as food, health, biotechnology and biofuels.

The mutagenesis may be performed by exposure of the parent strain of Chlorella microalgae to a mutagenic chemical for a specific time. The exposure time to a given concentration of the mutagenic chemical also influences its lethality. In other words, the concentration (and quantity) of the mutagenic chemical used for performing the mutagenesis, combined with the exposure time, can determine the amount of mutation undergone by the organism. Optionally, the specific time for treatment with the mutagenic chemical is 1 to 120 minutes. More optionally, the quantity of the mutagen (or mutagen dose) is defined as a concentration of the mutagen multiplied by an exposure time. Specifically, the sub-lethal quantity of the mutagen is obtained by altering the mutagen concentration, the exposure time, or a combination of both, for example.

It will be appreciated that heavily mutagenized cells of the organism accumulate multiple mutations of genetic material, which are often deleterious to the viability or overall fitness of the mutagenized strain as assessed using standard growth performance assays, for example. This is of particular importance in a haploid organism such as Chlorella microalgae, where only a single copy of each gene is present. It is common that multiple mutations occur within the genomes of mutagenized strains. Use of a high quantity of alkylating agent for performing the mutagenesis may result in point mutations that create aberrations in the mutated strain of Chlorella microalgae as compared to the parent strain of Chlorella microalgae, or may result in death of the mutated strain of Chlorella microalgae. Optionally, the degree of mutagen kill may be measured by determining cell viability using a conventional quantification technique (for example, viable counts, viability staining, flow cytometry, and the like) known in the art.

Therefore, using a sub-lethal or non-lethal quantity (0.1 to 2.0 M) of the mutagenic chemical, such as alkylating agents, for a specific time, enables generation of desired phenotypes while preventing or minimising accumulation of undesirable traits that might reduce overall strain fitness, hamper growth, or result in death of the organism. Typically, optimal mutagen dose is determined empirically for a specific species of an organism, and varies from organism to organism. Similarly, different mutagens have different mechanisms of action, and an optimal dosing strategy (i.e. relative concentration multiplied by time) using a mutagen for the target organism can be determined for each. Therefore, besides determining the dosing strategy, identification of a suitable mutagen is equally essential as each mutagen class has a specific mechanism of action that directly affects the diversity of mutations generated in the target organism which, thereby, also impacts the utility of the resulting mutant library.

In an example, the mutagenesis of the parent strain of Chlorella microalgae may be performed by exposure of the parent strain of Chlorella microalgae to a 1.0 M dose of EMS for an exposure time of 1 minute or a dose of EMS above 1.0 M for exposure time of a shorter period, for example 30 seconds, to produce a 50% lethality to the Chlorella microalgae species, for example. This process, i.e. combining mutagenic chemical concentration and exposure time to said mutagen, results in a kill rate which acts as a proxy for mutation frequency. Consequently, surviving cells of said process have one or more mutations within their genomes. Collectively, such cells comprise a pool (or library) of mutations from which can be selected desirable variant strains using a suitable method.

Alternatively, mutagenesis is performed by exposure of the parent strain of Chlorella microalgae to a physical mutagen, optionally wherein the physical mutagen comprises at least one of UV light, gamma rays, X-rays. These mutagens cause changes in the genotype of the parent strain of Chlorella microalgae to result in the mutated strain of Chlorella microalgae. In such an instance, as an alternative to performing the mutagenesis of the parent strain of Chlorella microalgae by exposure to the mutagenic chemicals, mutagenesis by exposure to physical mutagens can be performed to obtain the mutated strain of Chlorella microalgae.

Beneficially, mutagenesis and, in particular, the use of a mutagenic chemical, preferably sub-lethal quantities thereof, according to the invention, results in genetic variant strains of Chlorella microalgae in which the overall chlorophyll content, chitin content, protein content and high digestibility thereof as disclosed of the strain is the result of a stable genetic mutation.

In an embodiment, the strain of Chlorella microalgae is genetically stable. The term “genetically stable” as used herein, refers to a characteristic of a species or a strain/isolate to resist changes and maintain its genotype over multiple generations or cell divisions, ideally hundreds to several thousand generations, in non-selective conditions.

Notably, the parent strains of Chlorella microalgae are haploid. A haploid parent strain prevents the variant strains thereof from reverting back from a desired genotype to the genotype commonly associated with the parent strain of Chlorella microalgae over successive generations of cultivation, beneficially exhibiting relative stability of the desired phenotype in such strains. The strain of Chlorella microalgae (i.e. the variant strain of Chlorella microalgae) is genetically stable. Notably, the quantitative analysis, including flow cytometry, or optionally, qualitatively, confocal microscopy, of variant strains of Chlorella microalgae maintained both on agar and in liquid culture is sufficient to conclude that the phenotype, such as reduced chlorophyll, is genetically stable in the variant strain of Chlorella microalgae. Further, the stability of genetic mutations can also be confirmed by direct genetic sequencing.

In an embodiment, the Chlorella microalgae of the invention is cultivated in a heterotrophic growth mode. Notably, algae such as Chlorella microalgae can grow in conditions ranging from optimal to extreme and in varied habitats. In a preferred embodiment, the variant strains of the invention are cultivated in the heterotrophic growth mode (i.e. cultivatable solely on an organic carbon energy source, such as glucose, in the absence of light). Beneficially, such heterotrophic growth allows large scale economical production of the variant strains as a result of the superior growth rate and biomass yield that can be produced in proven existing plant designs, when compared to phototrophic or mixotrophic methods of microalgal cultivation.

Preferably, Chlorella microalgae of the invention are produced by a food-grade process to deliver a food-grade product. The Chlorella microalgae are cultivated in fermentation medium using a heterotrophic production process; in which an organic carbon energy source, preferably glucose, is used as feedstock and is supplied either in batch mode or preferably, fed-batch mode. Beneficially, the fed-batch process can deliver a higher final DCW and normally results in higher biomass productivities and consequently, a faster process. Optionally, the glucose feeding process could be continuous, providing an accurate feeding profile can be achieved. Such a feeding profile takes into account a feed rate, a current growth rate of the culture and a target media glucose concentration to achieve optimal growth of the low chlorophyll, high protein Chlorella variants. Moreover, calculations for the rate of feed of glucose are standard and known in the art. Optionally, the fermentation can also operate in semi-continuous mode with several draws, providing that nutrients are added to compensate for the broth removal rate. The process will start in batch mode (“the seed train”); being fed one or multiple glucose boluses as required to achieve the desired biomass density that is required to inoculate the main fed-batch production process. Specifically, the process will start with a 1 mL vial of Chlorella microalgal biomass frozen at −80° C. that, when thawed, inoculates a seed train that takes place in two phases (P1 and P2) in sterile baffled and ventilated Erlenmeyer flasks. After completion of the seed train, the production fermenter is inoculated with the required volume to produce an initial concentration of at least 0.8 grams per litre, or, in an alternative embodiment, of at least 3 grams per litre.

In an embodiment, the strain of Chlorella microalgae is cultivated:
at a specific temperature;
for a predefined period of time;
without the presence of light; and
in the presence of an organic carbon energy source.

In an embodiment, the specific temperature is in a range of 20 to 35° C., optionally in the range of 26 to 29° C., e.g. about 28° C.

In an embodiment, the specific temperature is in a range of 20 to 35° C., optionally in the range of 25 to 30° C., optionally in the range 28° C. to 30° C., e.g. about 29° C.

In an embodiment, the predefined period of time is in a range of 1 to 5 weeks, optionally in the range of 1 to 3 weeks. Alternatively, the predefined period of time is in the range of 1 to 7 days, optionally in the range of 1 to 5 days.

In an embodiment, the organic carbon energy source is glucose and/or acetate, preferably glucose. Optionally, the organic carbon energy source is derived from invert sugar, where the enzyme invertase has been used to hydrolyse sucrose into glucose and fructose.

With the process as hereinbefore described, biomass densities that are typically greater than 60, 65, 70, 75, 80, 85, 90, 95, 100, 105, 100, 115, 120, 125, 130, 135, 140, 145, 150, 155, or 160 g/L can be achieved. A higher final biomass density could be achieved by running a longer fermentation process, or using a continuous feed profile, for example. After the fermentation process, the biomass is optionally washed with water and/or concentrated to, for example, 200 g/L by centrifugation. Additionally, optionally, the biomass is then lysed by mechanical means to crack or break open the cells. The processed biomass is then spray dried. After spray drying, the powder is packed promptly to avoid moisture increase and oxidative phenomena.

In an embodiment, the organic carbon energy source is glucose having a glucose to biomass conversion ratio of more than 0.45. It will be appreciated that the high protein and low starch variant strains of Chlorella microalgae require a controlled concentration of glucose in fermentation broth to maintain optimal (or economical) growth, thereof. The conversion of glucose to biomass as a result of cultivating Chlorella microlagae in the fermentation broth is typically measured as a glucose to biomass conversion ratio or glucose to biomass yield coefficient. Moreover, said variants of Chlorella microalgae require a continuous feeding throughout the fermentation run, in order to sustain optimal growth by maintaining the optimal glucose level in the fermentation broth (as described above). In this regard, a glucose to biomass conversion ratio of >0.45, such as 0.55, is preferred. To achieve this, an automated feed profile may be developed to maintain a target of 20 g/L optimal glucose concentration in the fermentation broth for the said strain. It will be appreciated that the optimal glucose level can vary for different species of Chlorella microalgae. For example, variants of Chlorella sorokiniana with a low starch phenotype may exhibit optimal growth and glucose to biomass conversion ratio at circa 10 g/L glucose, but exhibit growth inhibition at circa 30 g/L glucose concentration in the fermentation broth. By contrast, a low starch variant of Chlorella vulgaris does not show decline in growth until a concentration of 60-70 g/L glucose in the fermentation broth. Sub-optimal growth of Chlorella microalgae resulting from the non-optimised feeding or concentration of glucose typically also results in a reduction in the glucose to biomass yield coefficient. Therefore, it will be appreciated that an optimal glucose feed regime for the particular Chlorella microalgae is required throughout the fermentation to achieve efficient, economic and timely conversion of glucose to biomass.

Typically, the variant strain of Chlorella sorokiniana is cultivated at a specific temperature, optionally ranging from 20 to 35° C. and more optionally in a range from 28 to 30° C., for a predefined period of time, such as in a range of 1 to 5 weeks, optionally in a range of 1 to 3 weeks, more optionally less than 7 days, optionally without the presence of light, i.e. in the dark or absence of light, and in the presence of an organic carbon energy source such as for example glucose (heterotrophic growth mode) and/or acetate (mixotrophic growth mode).

A third aspect of the invention provides a Chlorella microalgae comprising a genomic DNA sequence that is at least 50% identical to Sequence 2 (SEQ ID NO: 2). The genomic DNA sequence Sequence 2 (SEQ ID NO: 2) encodes for the ChII subunit of magnesium chelatase. Preferably the genomic DNA sequence is at least 60% identical to Sequence 2 (SEQ ID NO: 2), more preferably at least 70% identical to Sequence 2 (SEQ ID NO: 2), still more preferably at least 80% identical to Sequence 2 (SEQ ID NO: 2). In some particularly preferred embodiments, the genomic DNA sequence is at least 85% identical to Sequence 2 (SEQ ID NO: 2), such as at least 80% identical to Sequence 2 (SEQ ID NO: 2), at least 85% identical to Sequence 2 (SEQ ID NO: 2), at least 90% identical to Sequence 2 (SEQ ID NO: 2), or at least 95% identical to Sequence 2 (SEQ ID NO: 2).

In one aspect, the invention provides a strain of Chlorella microalgae comprising a genomic DNA sequence that is at least 50% identical to Sequence 4 (SEQ ID NO: 4). The genomic DNA sequence Sequence 4 (SEQ ID NO: 4) encodes for a phytoene desaturase. Preferably the genomic DNA sequence is at least 60% identical to Sequence 4 (SEQ ID NO: 4), more preferably at least 70% identical to Sequence 4 (SEQ ID NO: 4), still more preferably at least 80% identical to Sequence 4 (SEQ ID NO: 4). In some particularly preferred embodiments, the genomic DNA sequence is at least 85% identical to Sequence 4 (SEQ ID NO: 4), such as at least 80% identical to Sequence 4 (SEQ ID NO: 4), at least 85% identical to Sequence 4 (SEQ ID NO: 4), at least 90% identical to Sequence 4 (SEQ ID NO: 4), or at least 95% identical to Sequence 4 (SEQ ID NO: 4), more preferably at least 99% identical to Sequence 4 (SEQ ID NO: 4) and still more preferably it is identical to Sequence 4 (SEQ ID NO: 4).

In one aspect, the invention provides a strain of Chlorella microalgae comprising a genomic DNA sequence that is at least 50% identical to Sequence 5 (SEQ ID NO: 5). The genomic DNA sequence Sequence 5 (SEQ ID NO: 5) encodes for a phytoene desaturase. Preferably the genomic DNA sequence is at least 60% identical to Sequence 5 (SEQ ID NO: 5), more preferably at least 70% identical to Sequence 5 (SEQ ID NO: 5), still more preferably at least 80% identical to Sequence 5 (SEQ ID NO: 5). In some particularly preferred embodiments, the genomic DNA sequence is at least 85% identical to Sequence 5 (SEQ ID NO: 5), such as at least 80% identical to Sequence 5 (SEQ ID NO: 5), at least 85% identical to Sequence 5 (SEQ ID NO: 5), at least 90% identical to Sequence 5 (SEQ ID NO: 5), or at least 95% identical to Sequence 5 (SEQ ID NO: 5), more preferably at least 99% identical to Sequence 4 (SEQ ID NO: 4) and still more preferably it is identical to Sequence 5 (SEQ ID NO: 5).

In one aspect, the invention provides a strain of Chlorella microalgae comprising a genomic DNA sequence that is at least 50% identical to Sequence 7 (SEQ ID NO: 7). The genomic DNA sequence Sequence 7 (SEQ ID NO: 7) encodes for a ChIH subunit of a magnesium chelatase. Preferably the genomic DNA sequence is at least 60% identical to Sequence 7 (SEQ ID NO: 7), more preferably at least 70% identical to Sequence 7 (SEQ ID NO: 7), still more preferably at least 80% identical to Sequence 7 (SEQ ID NO: 7). In some particularly preferred embodiments, the genomic DNA sequence is at least 85% identical to Sequence 7 (SEQ ID NO: 7), such as at least 80% identical to Sequence 7 (SEQ ID NO: 7), at least 85% identical to Sequence 7 (SEQ ID NO: 7), at least 90% identical to Sequence 7 (SEQ ID NO: 7), or at least 95% identical to Sequence 7 (SEQ ID NO: 7), more preferably at least 99% identical to Sequence 7 (SEQ ID NO: 7) and still more preferably it is identical to Sequence 7 (SEQ ID NO: 7).

In one aspect, the invention provides a strain of Chlorella microalgae comprising a genomic DNA sequence that is at least 50% identical to Sequence 25 (SEQ ID NO: 25). The genomic DNA sequence Sequence 25 (SEQ ID NO: 25) encodes for a ChIH subunit of a magnesium chelatase. Preferably the genomic DNA sequence is at least 60% identical to Sequence 25 (SEQ ID NO: 25), more preferably at least 70% identical to Sequence 25 (SEQ ID NO: 25), still more preferably at least 80% identical to Sequence 25 (SEQ ID NO: 25). In some particularly preferred embodiments, the genomic DNA sequence is at least 85% identical to Sequence 25 (SEQ ID NO: 25), such as at least 80% identical to Sequence 25 (SEQ ID NO: 25), at least 85% identical to Sequence 25 (SEQ ID NO: 25), at least 90% identical to Sequence 25 (SEQ ID NO: 25), or at least 95% identical to Sequence 25 (SEQ ID NO: 25), more preferably at least 99% identical to Sequence 25 (SEQ ID NO: 25) and still more preferably it is identical to Sequence 25 (SEQ ID NO: 25).

In one aspect, the invention provides a strain of Chlorella microalgae comprising a genomic DNA sequence that is at least 50% identical to Sequence 66 (SEQ ID NO: 66). The genomic DNA sequence Sequence 66 (SEQ ID NO: 66) encodes for a ChII subunit of a magnesium chelatase. Preferably the genomic DNA sequence is at least 60% identical to Sequence 66 (SEQ ID NO: 66), more preferably at least 70% identical to Sequence 66 (SEQ ID NO: 66), still more preferably at least 80% identical to Sequence 66 (SEQ ID NO: 66). In some particularly preferred embodiments, the genomic DNA sequence is at least 85% identical to Sequence 66 (SEQ ID NO: 66), such as at least 80% identical to Sequence 66 (SEQ ID NO: 66), at least 85% identical to Sequence 66 (SEQ ID NO: 66), at least 90% identical to Sequence 66 (SEQ ID NO: 66), or at least 95% identical to Sequence 66 (SEQ ID NO: 66), more preferably at least 99% identical to Sequence 66 (SEQ ID NO: 66) and still more preferably it is identical to Sequence 66 (SEQ ID NO: 66).

In one aspect, the invention provides a strain of Chlorella microalgae comprising a genomic DNA sequence that is at least 50% identical to Sequence 27 (SEQ ID NO: 27). The genomic DNA sequence Sequence 27 (SEQ ID NO: 27) encodes for a phytoene desaturase. Preferably the genomic DNA sequence is at least 60% identical to Sequence 27 (SEQ ID NO: 27), more preferably at least 70% identical to Sequence 66 (SEQ ID NO: 27), still more preferably at least 80% identical to Sequence 27 (SEQ ID NO: 27). In some particularly preferred embodiments, the genomic DNA sequence is at least 85% identical to Sequence 27 (SEQ ID NO: 27), such as at least 80% identical to Sequence 27 (SEQ ID NO: 27), at least 85% identical to Sequence 27 (SEQ ID NO: 27), at least 90% identical to Sequence 27 (SEQ ID NO: 27), or at least 95% identical to Sequence 27 (SEQ ID NO: 27), more preferably at least 99% identical to Sequence 27 (SEQ ID NO: 27) and still more preferably it is identical to Sequence 27 (SEQ ID NO: 27).

A fourth aspect of the invention provides an algae biomass derived from the Chlorella microalgae as hereinbefore described.

A fifth aspect of the invention is a protein isolate or concentrate derived from an algae biomass, wherein the algae biomass is as hereinbefore described, or is derived from the strain of Chlorella microalgae as hereinbefore described.

The term “protein isolate” is used to describe a refined form (typically the most highly refined form) of protein product that is separated from other biomass components by physical or chemical means. It contains the greatest concentration of protein at typically 90% by dry weight and substantially no dietary fibre.

The term “protein concentrate” is used to describe refined protein products that are less concentrated than protein isolates, as they contain residual carbohydrate and dietary fibre. Accordingly, protein concentrates typically comprise 80% protein by dry weight.

Protein isolates and protein concentrates are produced by such methods as: wet extraction (alkali extraction/isoelectric precipitation), dry fractionation (air classification), salt extraction, micellization and mild fractionation. The efficiency of the extraction process depends on the physiochemical properties of the starting material, in addition to the method and conditions (such as pH, temperature, time of treatment etc) applied. Furthermore, it is known in the art that the physiochemical and functional properties of protein extracts; such as: emulsifying, foaming and gelling properties, in addition to solubility, water holding capacity, oil holding capacity, flavour, texture, digestibility, hydrophobicity and the like, can be modified or enhanced by physical, chemical or biological processes to improve their function and application as a food ingredient. Examples of such physical modification processes include: high-pressure treatment, heat with sheer treatment (extrusion), cold atmospheric pressure plasma treatment and ultrasonic treatment. Examples of such chemical modification processes include: glycation, acylation and deamidation. Examples of such biological modification processes include: fermentation and enzymatic modification (Shanthakumar et al. 2022; DOI: 10.3390/molecules27165354).

A sixth aspect of the invention provides a composition comprising an algae biomass as hereinbefore described, or a protein isolate or concentrate as hereinbefore described, employed in at least one of: human foods, human nutraceutical preparations or formulations, animal feeds, pharmaceutical compositions including vaccines, cosmetics, personal care compositions, personal care devices.

In an embodiment, the composition may be employed in at least one of: human foods, human nutraceutical preparations or formulations, animal feeds, pharmaceutical compositions including vaccines, cosmetics, personal care compositions, personal care devices.

The term “food” refers to an edible product that can be directly or indirectly (such as, subsequent to preparation) consumed by humans and/or animals. The term “food ingredient” refers to a substance incorporated into food during one of: production, processing, treatment, packaging, transportation, distribution, preservation, storage and so forth of food. Optionally, the food ingredients are incorporated into the food to improve and/or maintain freshness, nutritional value, appearance, texture, taste and safety of the food.

The non-genetically modified and non-transgenic Chlorella microalgae biomass is suitable for direct incorporation into food products, whole or as an ingredient. Food products include, but are not limited to, bakery products, microalgae flour, pasta, rice, breakfast cereals, cereal bars, confections, sauces, soups, dairy substitutes, frozen desserts, ice creams, yoghurts, smoothies, creams, spreads, salad dressings, mayonnaises, food garnishing and seasoning, candies, gums, jellies, beverages, snacks, plant-based meat analogues, plant-based fish and seafood, infant formulas, plant-based egg substitutes.

Optionally, provided, is a method of using the aforementioned composition as an ingredient in at least one of: human foods, human nutraceutical preparations or formulations, animal feeds, pharmaceutical compositions including vaccines, cosmetics, personal care compositions, supplements, personal care devices or textiles, dyes, inks or for the production of fuels. The method of use comprises using the algae biomass ingredient comprising the variant strain of Chlorella microalgae as any one of: a dried powder, dried flakes, a frozen paste, an extract (protein isolate or protein concentrate), solutions, suspensions, solution preconcentrates, emulsions, emulsion pre-concentrates, a concoction, tablets, pills, pellets, capsules, caplet, concentrates, granules, and so forth. Furthermore, a dried, fresh, or frozen part of the Chlorella microalgae, a protein concentrate, a protein isolate, oil derived from the Chlorella microalgae, a homogenate, whole cell, lysed cell and so forth can be used in preparation of human foods, human nutraceutical preparations or formulations, animal feeds, pharmaceutical compositions, cosmetics, personal care compositions, personal care devices and fuels. Moreover, the Chlorella microalgae can be used to prepare compositions in any way known to the skilled person.

Various embodiments and variants disclosed above apply mutatis mutandis to the composition.

A seventh aspect of the invention provides a method of producing a Chlorella microalgae, the method comprising:
a) obtaining a parent strain of Chlorella microalgae;
b) performing mutagenesis of the parent strain of Chlorella microalgae;
c) cultivating the mutated strain of Chlorella microalgae at a specific temperature, for a predefined period of time, and in the presence of an organic carbon source; and
d) identifying and isolating mutants of the parent strain of Chlorella microalgae having a mutation in at least one gene that encodes for phytoene desaturase or a subunit thereof, and further having a mutation in at least one gene that encodes for magnesium chelatase or a subunit thereof.

The modified strains of Chlorella may be subjected to additional rounds of mutagenesis.

Optionally, the method further comprises performing steps (b) to (d) repeatedly for selecting healthy colonies of the modified strain of Chlorella based on desired traits, wherein the desired traits comprise a colour, a pigment content, a protein content, a cell wall modification and improved tolerance to process conditions selected from a group of temperature, pH, sheer stress and osmolality. Furthermore, the Chlorella strains are stable through generations. Various embodiments and variants disclosed above apply mutatis mutandis to the method.

In this regard, after mutagenesis, the target strains of Chlorella microalgae are isolated after cultivation under suitable growth condition, preferably cultivated in a heterotrophic growth mode at a specific temperature ranging from 20 to 35° C., optionally, in a range from 25 to 28° C., for a predefined period of time ranging from 1 to 5 weeks, optionally in a range of 1 to 3 weeks, more optionally less than 7 days, and in the presence of an organic carbon source such as for example glucose and/or acetate, without the presence of light, i.e. in the dark or in the absence of light. Isolation of suitable variants may be performed by any means known to the skilled person.

Flow cytometry (FCM) is a technique for detecting and measuring physical and chemical characteristics of a sample containing cells or particles. For instance, the fluorescence intensity of cells or particles stained with calcofluor white or optionally lectins conjugated to fluorophores is correlated with the chitin content of the cells or particles, respectively. The sample containing cells or particles are often labelled with fluorescent markers for analysing cells and components. Flow cytometry is based upon analysis of the relative signal strength of fluorescence of a sample (autofluorescence) or fluorescent marker bound to a sample containing cells or particles. Optionally, flow cytometry serves as an enrichment step of physically sorting (namely, separating and isolating) desired cells away from cells with a parental phenotype and thereby purifying cells of interest based on their specific optical properties, referred to as fluorescence-activated cell sorting or cell sorting by flow cytometry. Optionally, such isolated cells are expanded by cultivation and re-sorted through one or more additional rounds of flow cytometry to confirm the stability of the phenotype or to enrich for a secondary mutant phenotype, for example a chlorophyll-deficient phenotype, or a colour phenotype, according to the fluorescence parameters chosen. They can then be further expanded in liquid culture or plated onto agar plus glucose plates for scoring of colours with respect to other mutations.

In an embodiment, the identification of the strain of Chlorella microalgae of the invention comprises sorting or screening the cells by any suitable technique, such as by using flow cytometry. The variant strain of Chlorella microalgae may be selected for example based on a desirable pigment content, wherein the desirable pigment content is based upon a relative signal obtained on cell sorting by flow cytometry. The use of flow cytometry provides the advantages of examining thousands of cells per second and in real time and processing quantifiable data over a computer coupled to a flow cytometer. Furthermore, flow cytometry helps in cell counting, cell sorting, determining cell characteristics and function and detecting microorganisms.

The method further comprises selecting healthy (or viable) cells or filtering out unhealthy cells of the variant strain of Chlorella microalgae, preferably by cultivation under non-permissive or stressful conditions. It will be appreciated that during mutagenesis of the parent strain of Chlorella microalgae, cells of the Chlorella microalgae may acquire mutations at multiple sites within the genome, including a mutation or mutations that are causative for the desired phenotype. However, some mutated cells (strains) of Chlorella microalgae may additionally acquire deleterious mutations as a consequence of exposure to the mutagenic agent, resulting in one or more undesired mutations, for instance in essential genes. In such an instance, it is essential to filter out these unhealthy cells of the Chlorella microalgae associated with the deleterious mutations, to ensure selection of only those cells which are robust and able to grow well under desired cultivation conditions. This can be achieved by cultivation of the mutated strains during the period immediately following exposure to the chemical or physical mutagen under stressed or less permissive (or non-permissive) conditions, for instance at the limit of, or slightly above the normal upper temperature for cultivation and in the absence of light but in the presence of glucose. Optionally, mutated strains are cultivated under phototrophic conditions, more optionally, mutated strains are cultivated under mixotrophic conditions. Only robust strains are able to proliferate under stressful conditions. This approach enriches for strains that are not compromised in their general growth characteristics. Furthermore, after cultivation, the desired phenotypes related to reduced chlorophyll or chitin content can be scored. Undesired phenotypes, including chlorophyll or chitin content at levels associated with the parent strain or the wild-type strains of Chlorella microalgae, are not selected. In other words, they are filtered out. Optionally, cells of Chlorella microalgae that exhibit the desired phenotype across a series of generations are selected as healthy cells. More optionally, the mutated strain of Chlorella microalgae is cultivated at a temperature that is slightly higher than an ideal temperature for cultivation of the microalgal strain, to select only healthy cells of the Chlorella microalgae.

Optionally, the method comprises recovering the mutant strains of Chlorella microalgae on a solid agar plate. Recovering the mutant strains of Chlorella microalgae on the solid agar plate ensures isolation of only the viable cells for use in later steps of isolation of variant strains of Chlorella microalgae. Preferably, the mutant strains are sub-cultured several times on the solid agar plates to ensure they are free from a potential contamination from bacteria or fungi. Flow cytometry can be used to determine the chitin content of the variant strains in a quantitative manner, as described herein above.

Optionally, the method further comprises repeating, several times, mutagenesis and strain selection of the parent strain of Chlorella microalgae. The said repetition of mutagenesis, cultivation and isolation steps enables selecting healthy cells of the variant strains of Chlorella microalgae based on desired phenotypes (or traits) such as reduced chlorophyll content, preferably a combination of such phenotypes for example reduced chitin content, desirable colours, a pigment content, a high protein content or improved tolerance to process conditions. Incubating the library for a number of generations following mutagenesis is a useful strategy for removing viable, but undesirable genetic mutations which adversely affect overall cell performance, or “fitness”. Such a method permits the “stacking” of desirable traits in a Chlorella microalgae in a controlled manner. This is a significant improvement over prior art methods involving random mutagenesis followed by selection of desired mutants (e.g. desired phenotypes) as it is a directed process that does not rely on a random occurrence of a desired combination of mutations in a Chlorella microalgae.

Beneficially, the variant strain of Chlorella microalgae, exhibiting a chlorophyll content of less than 0.5 mg/g dry-cell weight, as a result of the described mutations in phytoene desaturase and magnesium chelatase, is a potential ingredient in various food and personal care applications. Furthermore, the reduced chlorophyll content of the variant strain of Chlorella microalgae is also associated with reduction in the unpleasant colour, smell and taste (organoleptics) associated with the wild-type strain of Chlorella microalgae, when used in the food and personal care applications. Additionally, beneficially, the variant strain of Chlorella microalgae having the reduced chlorophyll content can be incorporated at a higher percentage as an ingredient in food compositions, compared with the wild-type, as a result of such improvements in the organoleptic properties, which gives the chlorophyll-reduced Chlorella a neutral flavour.

The Chlorella microalgae biomass of the invention is a suitable ingredient in the production of texturized vegetable protein (TVP) or similar meat analogue or meat extender (products typically produced by extrusion) owing to its improved organoleptic properties and desirable colour.

Other uses include nutraceuticals, nutritional supplements (for example, nutritional supplements, hormone tablets, digestive capsules, tablets, powders, oils and the like) and animal feed. Additionally, the other uses of the algal biomass include cosmetics (for example, in lipsticks, powders, creams, exfoliants, facial packs, and so forth), personal care compositions and personal care devices (for example toothpastes, mouthwash, hand-wash, body-wash, body soaps, shampoos, oils, sun-creams, after-sun creams, sunblock and so forth), colourants. Further uses include pharmaceuticals (such as vaccines, various bioactives and delivery routes for other recombinant proteins and enzymes).

An eighth aspect of the invention provides a Chlorella microalgae strain selected from the following:
(i) a Chlorella vulgaris strain designated WC03, deposited on Dec. 14, 2023 at the Culture Collection of Algae and Protozoa (CCAP), SAMS Ltd., Scottish Marine Institute, OBAN, Argyll, PA37 1QA, United Kingdom, in accordance with the Budapest Treaty, with a Patent Deposit Designation of CCAP 211/143;
(ii) a Chlorella sorokiniana strain designated CS174, deposited on Dec. 14, 2023 at the Culture Collection of Algae and Protozoa (CCAP), SAMS Ltd., Scottish Marine Institute, OBAN, Argyll, PA37 1QA, United Kingdom, in accordance with the Budapest Treaty, with a Patent Deposit Designation of CCAP 211/142.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF DRAWINGS

One or more embodiments of the invention will now be described, by way of example only, with reference to the accompanying drawings, in which:

FIG. 1 is an illustration of steps of a method of producing a variant strain of Chlorella microalgae as hereinbefore described.

FIG. 2 shows that 4TC3/16 is a wild-type strain of Chlorella vulgaris that is taxonomically identical to the culture collection type strain of Chlorella vulgaris 211/11b. Culture collection type-strains of Chlorella vulgaris 211-11b and 211-11p, and Algenuity proprietary strain 4TC3/16 (4TC3) form a distinct clade amongst the collated green algae ITS2 sequences shown, demonstrating the taxonomic similarity between these isolates and confirming the designation of strain 4TC3 as Chlorella vulgaris. To achieve this conclusion, ITS2 genetic sequences of Parachlorella kessleri and Chlamydomonas reinhardtii, in addition to those belonging to members of the Chlorella genus were downloaded from the ITS2 database (Schultz et al., 2006), with each species represented by a sequence selected at random with the exception of Chlorella vulgaris which is represented by 4TC3/16, 211-11b and 211-11p strains. All sequences were aligned using ClustalW (Madeira et al., 2019; DOI: 10.1093/nar/gkz268) and the alignment trimmed to remove overhanging sequences with JalView (v2.11.1.3, Waterhouse et al., 2009; DOI: 10.1093/bioinformatics/btp033). A Neighbour-joining tree was constructed using ClustalW2 and visualised using Interactive Tree Of Life (v5.7, Letunic & Bork, 2006; DOI: 10.1093/bioinformatics/btl529).

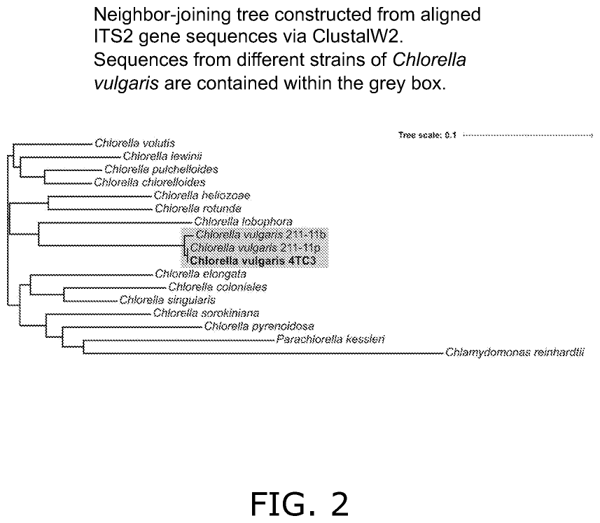

FIG. 3 shows the iteration of new Chlorella vulgaris variant WC03, achieved through successive rounds of chemical mutagenesis, originating from wild-type 4TC3/16.

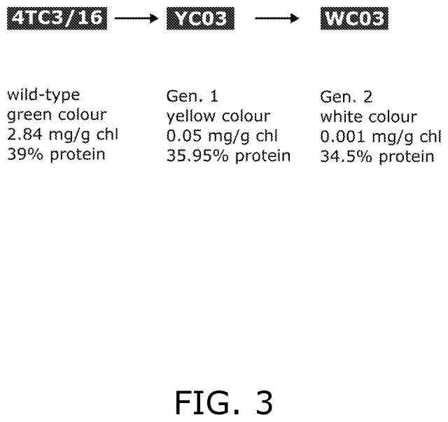

FIG. 4 shows the iteration of new Chlorella vulgaris variants, achieved through successive rounds of chemical mutagenesis, originating from wild-type 4TC3/16.

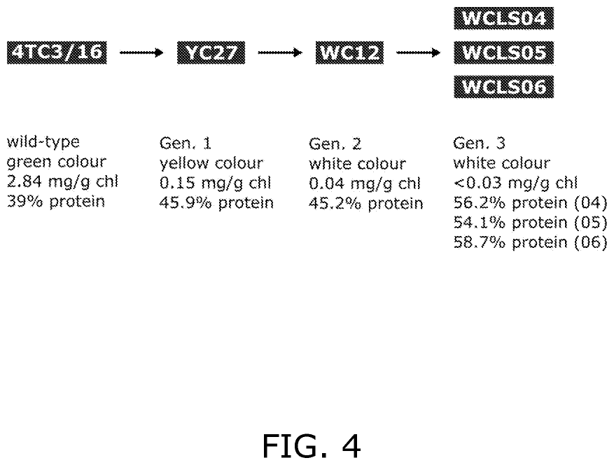

FIG. 5 shows the iteration of new Chlorella sorokiniana variants, achieved through successive rounds of chemical mutagenesis, originating from wild-type UTEX1230.

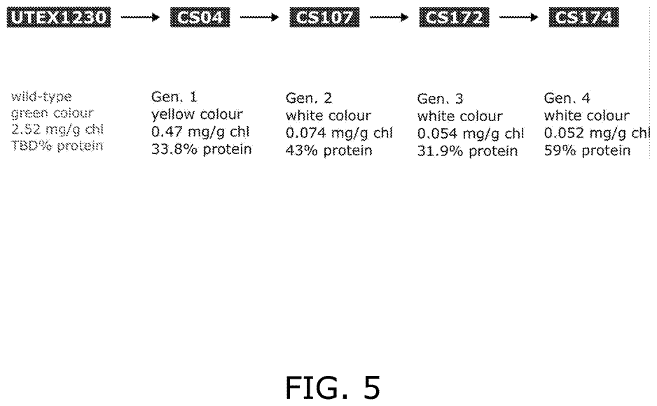

FIG. 6 shows a three-dimensional plot of CIELAB values of powdered samples prepared according to the method described herein. Strains affected by a mutation in magnesium chelatase subunit ChIH are designated by dark grey circle, Strains affected by a mutation in magnesium chelatase subunit ChII are designated by mid-grey circles, Comparative Example 1 is designated as a light-grey circle.

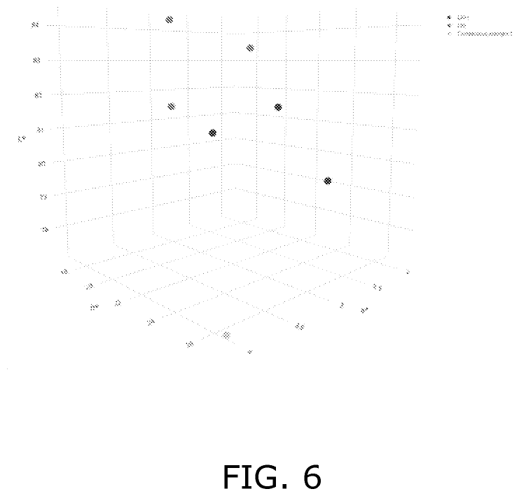

FIG. 7 shows a three-dimensional plot of CIELAB values of powdered samples prepared according to the method described herein.

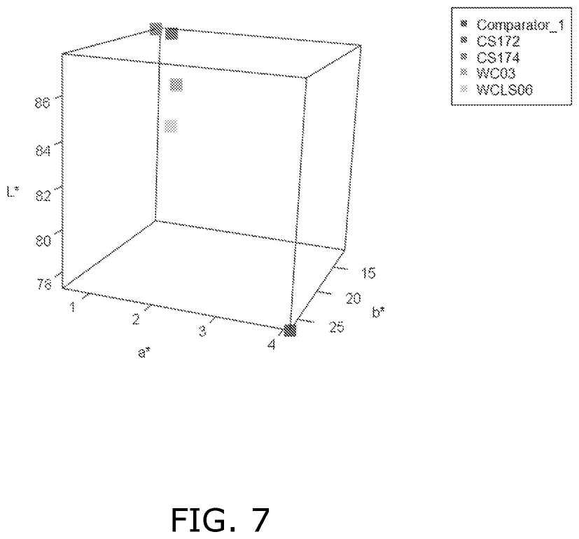

FIG. 8 shows a summary table of genetic variation in Chlorella vulgaris strains of the invention due to mutations. 1. Single Nucleotide Polymorphism (SNP) or Insertion/deletion (INDEL). 2. Effect of the mutation on the protein sequence derived from the translation of this gene. Missense variants result in a change to a single amino acid. Frameshift variants result in a disruption of the reading frame and the subsequent translation. Intron variants affect the intron regions. 3. Likelihood of this variant impacting the protein sequence and, therefore, potentially resulting in a phenotypic change, as identified using SnpEFF (Cingolani et al. 2012; DOI: 10.4161/fly.1969)

FIG. 9 shows a summary table of genetic variation in Chlorella sorokiniana strains of the invention due to mutations. 1. Single Nucleotide Polymorphism (SNP) or Insertion/deletion (INDEL). 2. Effect of the mutation on the protein sequence derived from the translation of this gene. Missense variants result in a change to a single amino acid. Frameshift variants result in a disruption of the reading frame and the subsequent translation. Intron variants affect the intron regions. 3. Likelihood of this variant impacting the protein sequence and, therefore, potentially resulting in a phenotypic change, as identified using SnpEFF (Cingolani et al. 2012; DOI: 10.4161/fly.1969)

### DETAILED DESCRIPTION

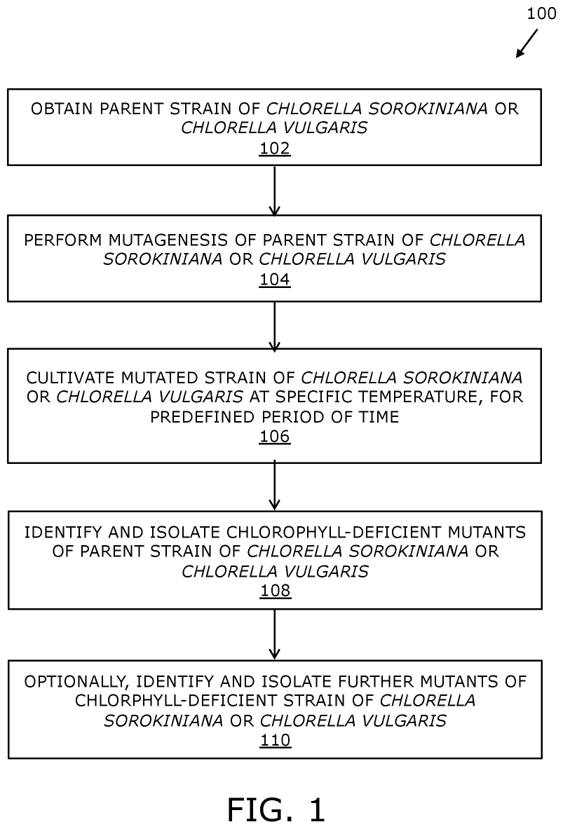

Referring to FIG. 1, shown is a flowchart 100 of steps of a method of producing a strain of Chlorella microalgae having an L* value in an L*a*b* colour space of greater than about 78. At step 102, a parent strain of Chlorella microalgae is obtained, such as from its natural habitat or a laboratory culture. At step 104, mutagenesis of the parent strain of Chlorella microalgae is performed. Herein, a mutagenic chemical such as an alkylating agent in its sublethal quantity and for a specific duration of time is used for mutagenesis of the obtained parent strain of Chlorella microalgae. The parent strain of Chlorella microalgae is subjected to mutagenesis in order to produce mutated, variant strains of Chlorella microalgae exhibiting a different phenotype, such as reduced chlorophyll content, high protein content, and so on, from that exhibited by the parent strain of Chlorella microalgae. Herein, mutagenesis is performed by exposing the obtained parent strain of Chlorella microalgae to EMS having a concentration in a range from 0.1 to 2.0 M for 1 to 120 minutes.

At step 106, the mutated strain of Chlorella microalgae is cultivated at a specific temperature, for a specific time, and in the presence of an organic carbon source. For example, the mutated strain of Chlorella microalgae is cultivated under heterotrophic growth mode using a source of carbon and energy, such as glucose, without any presence of light (i.e. in the dark or in the absence of light). In such case, as an example, the petri dishes containing the sample of Chlorella microalgae may be wrapped individually in a substantially opaque sheet, such as a foil, and then the wrapped-up petri dishes may be placed inside a cardboard box in the incubator. Other suitable ways of cultivating in the dark or without the presence of light can be used. Moreover, the heterotrophic growth of the mutated strain of Chlorella microalgae is achieved under suitable aseptic conditions.

Optionally, the mutated strain of Chlorella microalgae is obtained from a parent strain of Chlorella microalgae, cultivated using one or more of: a liquid or solid growth medium, including a fermentation medium containing an added carbon source such as glucose, or a mixotrophic growth medium containing acetate or a heterotrophic growth medium. In an example, the mutated strain of Chlorella microalgae is obtained from a parent strain of Chlorella microalgae, cultivated using a solid medium. Such a solid medium can be a regular agar plate. In such an instance, cells of the mutated strain of Chlorella microalgae are inoculated on agar plates at an appropriate cell density to achieve a dense biomass growth on the surface of the agar plates. The solid medium can be a high salt medium-glucose agar plate, wherein the high salt medium-glucose agar plate comprises: a growth medium such as High Salt Medium (HSM), glucose (for example, 1% w/v) and agar.

In another example, the mutated strain of Chlorella microalgae is cultivated using a liquid medium. Such a liquid medium can be at least one of TAP (Tris-Acetate-Phosphate), High Salt Medium (HSM), glucose (for example, having consistency of 1% w/v) and so forth. The fermentation medium comprises a source of nitrogen (such as proteins or nitrate or, more usually, ammonium), minerals (including magnesium, phosphorus, potassium, sulphur, calcium, and iron), trace elements (zinc, cobalt, copper, boron, manganese, molybdenum), an optional pH buffer, a source of carbon and energy (such as glucose, acetate) and so forth. Optionally, the parent strain of Chlorella microalgae is cultivated in a fermenter.

Furthermore, cultivation of the cells that have been exposed to mutagenesis at a higher than optimal cultivation temperature acts as a ‘stress’ filter such that only the more robust strains—where accumulated mutations have not produced a weakened or crippled organism can produce colonies on agar or viable daughter cells identified through a screen such as flow cytometry. As a result of cultivation of mutagenized cells at such elevated temperature or temperatures, fewer overall cells grow but those that do grow are more biologically and genetically fit with regard to growth and/or biomass production. Hence, those strains with reduced chlorophyll content that grow under these conditions and are scored based upon initial chlorophyll content should also be expected to be more robust with regard to application within an ultimate scalable commercially-relevant bioprocess. The repeated cultivation of the strains in the same growth conditions, i.e. heterotrophic growth conditions, produces generations of the variant strain of Chlorella microalgae with the desired phenotype compared to the starting mutant strain thereof.

At step 108, mutants of the parent strain of Chlorella microalgae having an L* value in an L*a*b* colour space of greater than about 78 are identified and isolated. Cells of the mutated strain of Chlorella microalgae having a phenotype different from the parent strain of Chlorella microalgae, are identified as the variant strain of Chlorella microalgae, and subsequently isolated for further application thereof. For example, when the mutated strain of Chlorella microalgae is cultivated using agar plates, colonies of the mutated strain of Chlorella microalgae on the agar plates that exhibit a different phenotype than the parent strain of Chlorella microalgae are identified as the variant strain of Chlorella microalgae. It will be appreciated that the mutated strains (or variants) are then selected based on one or more additional desirable phenotype, preferably reduced chlorophyll content, after growth on solid or liquid medium. Optionally, the phenotype is a scorable phenotype, wherein such phenotypes may be identifiable by various methods for such identification known to a person skilled in the art, such as, for example a relative signal obtained on cell sorting by flow cytometry. In a further example, the variant strains of Chlorella microalgae of the invention may be identified using L*a*b* CIELAB colour values and genetic sequencing of phytoene desaturate and magnesium chelatase genes.

The method may optionally include further step 110. At step 110, mutants of the parent strain of Chlorella microalgae having a protein content of at least 50% w/w are identified and isolated. Cells of the mutated strain of Chlorella microalgae having a phenotype different from the parent strain of Chlorella microalgae, are identified as the variant strain of Chlorella microalgae, and subsequently isolated for further application thereof. For example, when the mutated strain of Chlorella microalgae is cultivated using agar plates, colonies of the mutated strain of Chlorella microalgae on the agar plates that exhibit a different phenotype than the parent strain of Chlorella microalgae are identified as the variant strain of Chlorella microalgae. It will be appreciated that the mutated strains (or variants) are then selected based on one or more additional desirable phenotype, i.e. increased protein content, after growth on solid or liquid medium. Optionally, the phenotype is a scorable phenotype, wherein such phenotypes may be identifiable by various methods for such identification known to a person skilled in the art. In an example, the Chlorella microalgae is selected based on a desirable protein content, wherein the desirable protein content is based upon a relative signal obtained on cell sorting by flow cytometry or iodine staining, preferably by iodine staining.

The steps 102, 104, 106, 108, and 110 are only illustrative and other alternatives can also be provided where one or more steps are added, one or more steps are removed, or one or more steps are provided in a different sequence without departing from the scope of the claims herein. The method may, for example, further comprise repeating, several times, mutagenesis and strain selection of the parent strain of Chlorella microalgae. The said repetition of mutagenesis, cultivation and isolation steps enables selecting healthy cells of the variant strains of Chlorella microalgae based on desired phenotypes (or traits) such as an L* value in an L*a*b* colour space of greater than about 78, reduced chlorophyll content, and optionally, a combination of such phenotypes for example reduced chitin content, desirable colours, a pigment content, a high protein content or improved tolerance to process conditions. Incubating the library for a number of generations following mutagenesis is a useful strategy for removing viable, but undesirable genetic mutations which adversely affect overall cell performance, or “fitness”. Such a method permits the “stacking” of desirable traits in a Chlorella microalgae in a controlled manner.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

### Experimental Details

The following provides a detailed description of the preferred methodology for isolating and characterising Chlorella microalgae strains of the invention, in alignment with the specified embodiments.

### General Experimental Method for Obtaining, Identifying, and Isolating Chlorella Microalgae Strains

### Genetically Defining Chlorella Microalgae Strains Using PCR Amplification:

Chlorella microalgae was genetically defined by 18S and ITS2 sequencing as described above.

Fermentation medium (FERM) composition: glucose (111 mM), (NH4)2SO4 (47.7 mM), MgSO4·7H2O (2.8 mM), CaCl2·2H2O (204 μM), K2HPO4 (51.7 mM), NaH2PO4·H2O (63.3 mM), KOH (40 mM), citric acid (8.8 mM), H3BO3 (1.1 mM), Na2MoO4 (32 μM), ZnSO4·7H2O (974 μM), MnSO4·H2O (958 μM), NiCl2·6H2O (11 μM), FeSO4·7H2O (79.1 μM), CuSO4·5H2O (8 μM), Thiamine hydrochloride (5.65 μM), Biotin (92.1 nM), Cyanocobalamin (13.3 nM), D-Pantothenic acid (205.3 nM), 4-Aminobenzoic acid (656.3 nM)

The high salt medium (HSM) described herein comprised: NH4Cl (7 mM), MgSO4·7H2O (400 μM), CaCl2·2H2O (340 μM), K2HPO4 (4.13 mM), KH2PO4 (2.67 mM), Na2-EDTA (57.75 μM), (NH4) 6Mo7O24·4H2O (28.5 nM), Na2SeO3 (100 nM), ZnSO4·7H2O (2.5 M), MnCl2·4H2O (6 μM), Na2CO₃ (21.9 μM), FeCl3·6H2O (20 μM), CuCl2·2H2O (2 μM). Glucose is added at 1% (w/v) or 2% (w/v) or 3% (w/v) where indicated, in addition to: Thiamine hydrochloride (5.65 M), Biotin (92.1 nM), Cyanocobalamin (13.3 nM), D-Pantothenic acid (205.3 nM), 4-Aminobenzoic acid (656.3 nM) to produce HSM1GV or HSM2GV or HSM3GV, respectively.

### Mutagenesis Method to Obtain WC03 (Examples 1, 2 and 3), WC12 (Example 4), WCLS04 (Example 5), and WCLS06 (Example 6)

Chlorella microalgae strains were grown in 100 millilitres (ml) of nutrient rich liquid medium containing glucose or acetate (such as FERM or HSM as described herein) at a starting cell density of 2×10⁶ cells/ml. Cells were grown in the dark at 28° C. for 3-6 days, with 120 rpm agitation. Cell number was recorded during the incubation period using a haemocytometer and a light microscope. When cells are still in exponential phase of growth, reaching 1×10⁷ to 1×10⁸ cells/ml, an aliquot containing 1×10⁹ cells was harvested by centrifugation (4500 g for 10 minutes).

The resulting cell pellet was resuspended in 1 ml HSM media+1% glucose. An aliquot of ethyl methanesulphonate (EMS), with a resulting final concentration of 0.5M was added to the resuspended cells. The cells were incubated in the dark at 25° C. for 30-120 minutes. EMS is known to produce random mutations, such as nucleotide substitution, transition mutation, single nucleotide polymorphisms (SNPs) and the like, in the genetic makeup of the organism exposed thereto. The use of EMS may result in a mutated ethylguanine base in the DNA as a result of guanine alkylation. Repeated replication of such mutated DNA can result in a transition mutation, wherein original G: C base pairs change to A: T base pairs, thereby significantly changing the genetic makeup of the organism. In such case, the replication of such mutated DNA may create missense mutations or nonsense mutations within coding sequences or impacting gene expression or gene function by compromising regulatory sequence functionality including splice-site mutations.

After the mutation incubation period an aliquot of 30% sodium thiosulphate, in HSM+1% glucose media was added to the cell solution, with a final concentration of 5% sodium thiosulphate. The cells were incubated in the dark for a further 10 minutes. The sodium thiosulphate inactivates the alkylating agent and slows the rate of mutagenesis. After the inactivation incubation the cell solution was diluted in 25 ml of HSM 1% glucose media, centrifuged, and the supernatant discarded (into a 30% sodium thiosulphate solution). This wash step was repeated 2 more times to eliminate the residual EMS and stop the mutagenesis process.

After washing, the cells were resuspended in 50 ml of HSM+1% Glucose, TAP, or other nutrient rich media with a glucose or acetate carbon source. The cells were incubated in the dark, at 28° C., with 120 rpm agitation, for at least 24 hours. The cells were then plated on solid agar media (HSM+1% glucose or TAP) to isolate viable cell mutants derived from single cells. After 2-4 weeks the colonies were ready for phenotype selection. Alternatively, after 24 hours the mutant pool can be screened with flow activated cell sorting (flow cytometry), to isolate single cells with a desired phenotype, or a mutant pool enriched with the desired phenotype.

### Flow Cytometry Method to Isolate Chlorella Microalgae Strains

Cell sorting by flow cytometry was used as an enrichment step to sort chlorophyll-deficient cells away from wild-type cells based upon the relative signal strength of autofluorescence.

A sample containing cells was suspended in a fluid and injected into a flow cytometer instrument, wherein the flow of the sample was set at one cell at a time. The flow rate of the flow cytometer instrument was set to 60.000 to 500.000 events per minute.

The flow cytometer employs lasers of various wavelengths for multi-parametric analysis of the cells in a heterogenous cell population. The light scattered and fluoresced by the cell is a characteristic of the cell and components therein. A 488 or 561 nm laser was used to elicit strong chlorophyll autofluorescence from a mixture of live cells. The population of cells that exhibit strong autofluorescence was sorted away from those cells that have null or significantly reduced signal as an enrichment step to enrich for those cells within the total population that have accumulated mutations that knock down or abolish the chlorophyll signal. This step was applied between 2-7 days post-exposure to mutagen and was applied in liquid culture.

As a control to calibrate the cytometry, wild-type cells were extracted using 90% acetone to remove chlorophyll and were then photo-bleached using strong light for 20-30 minutes. These chlorophyll null cells were then used to calibrate the sorter with regard to chlorophyll deficient particles. Further, flow cytometry enables cell counting, cell sorting, determining cell characteristics and functions, detecting microorganisms, biomarker detection, protein engineering detection, and the like. Null cells including those desired cells with reduced chlorophyll content and expanded and resorted through one additional round to confirm the stability of the chlorophyll deficient phenotype. These cells can be further expanded in liquid culture or plated onto agar plus glucose plates for scoring of colour with respect to other mutants. The scoring method that was used to quantify and thereby directly compare the colour of chlorophyll-deficient Chlorella variants was the use of L*a*b* CIELAB colour values as enumerated, for example using the PCE-CSM 2 colourimeter (https://www.pce-instruments.com/) and the accompanying manufacturer's protocol.

Once expanded, the chlorophyll deficient cell population that is actively growing, can be sorted into sub-populations or single cells using the application of different lasers exciting at specific wavelengths and concurrent detection of deflection of the laser beam and specific fluorescence emissions of higher wavelength photons from cellular compounds, which can be used to differentiate specific pigment combinations that would ultimately influence the resultant stable biomass colour for a given biomass that is derived from a particular population of cells or single cells carrying specific genotype.

The method further comprised filtering out unhealthy colonies of the modified strain of Chlorella vulgaris. It will be appreciated that during mutagenesis of the wild-type strain of Chlorella vulgaris, cells of the modified strain of Chlorella vulgaris may acquire mutations at multiple sites within the genome, including a mutation or mutations that are causative for the desired phenotype. However, some colonies of the modified strain of Chlorella vulgaris may additionally acquire deleterious mutations corresponding to one or more undesired phenotypes, for instance in essential genes. In such an instance, it is essential to filter out these unhealthy colonies of the modified strain of Chlorella vulgaris associated with the deleterious mutations, to ensure selection of only those colonies that are robust and able to grow well under desired cultivation conditions. This was achieved by cultivation of the organisms during the period immediately following exposure to the chemical or physical mutagen under stressed or less permissive (or non-permissive) conditions, for instance at the limit of or slightly above the normal upper temperature for cultivation and in the absence of light but in the presence of glucose. Only robust strains are able to proliferate under these conditions. This approach enriches for strains that are not compromised in their replication capacity. Furthermore, after cultivation in the dark, the desired phenotypes related to colour can be scored using techniques described herein above.

In one such example, the desired phenotype of the modified strain of Chlorella vulgaris is associated with white, cream, pale yellow, yellow, pale green, golden, caramel, orange, red or lime colour. Undesired colonies will be associated with other colours, including the wild-type, dark green colour and are not selected. In other words, they are filtered out. Colonies of modified strains of Chlorella vulgaris that exhibit the desired phenotype across a series of generations were selected as healthy colonies. The mutated strain of Chlorella vulgaris is cultivated at a temperature that is slightly higher than an ideal temperature (i.e., above 28° C.) for cultivation of the microalgal strain, to select only healthy colonies of the modified strain of Chlorella vulgaris.

Colonies of the modified strains of Chlorella vulgaris associated with the desired phenotypes are isolated and streaked sequentially and iteratively on a solid medium to obtain pure colonies as well as to assess the stability of the colour phenotype under conditions more approximating a commercial cultivation scheme. The pure colonies were further inoculated using a liquid media. The liquid media was selected from one or more of TAP (Tris-Acetate-Phosphate), High Salt Medium (HSM) plus glucose (for example, having 1% w/v glucose).

The pure colonies were cultivated in dark conditions at the specific temperature of 25° C. for 1-3 weeks and monitored over multiple successive generations for stable phenotypes.

Single chlorophyll-deficient Chlorella microalgae (with a chlorophyll content below 0.5 mg/g dry cell weight) were isolated from a mixed mutant pool using flow cytometry. When light energy is absorbed by chlorophyll, part of the energy is used to drive photosynthesis via photochemical energy conversion, the remaining energy is lost as heat or emitted as fluorescence radiation. This fluorescence is also called chlorophyll autofluorescence.

When carrying out automated cell sorting, cells in solution are drawn into a flow cytometer and manipulated by fluidics into a separated single file cell stream (hydrodynamic focusing). Cells pass through the laser, where natural (such as chlorophyll) or artificial fluorophores are excited by this light and emit fluorescence with a specific wavelength spectrum. The fluorescent emission is detected by photomultiplier tubes (PMTs) or photodiodes. A voltage pulse (an event) is created when a change in the number of photons is detected by a PMT. The area of this pulse correlates to the fluorescence intensity (FI) of the fluorophore. This information is automatically collected by the machine and is displayed live on flow cytometry software.

The combination of mirrors, filters and detectors allows the machine to detect fluorescence at specific bands of wavelength. Commercially available lasers suitable for optimal excitation of chlorophyll are either 488 and 561 nm. The chlorophyll emission from this excitation ranges from 640 to 850 nm. In this example, the emission was monitored using a 695±40 nm dichromatic filter. Chlorophyll and additional pigments within the cell can also be excited by other lasers, including 349 nm, 355 nm, 405 nm, 445 nm, 532 nm, 594 nm, 640 nm, 740 nm, with emissions ranging from 350-850 nm.

The 695±40 nm FI of individual Chlorella cells was correlated to the chlorophyll content of the cells. Software controlling a flow cytometer allowed one or a series of custom gates to be created containing cells with specific fluorescent properties at different excitation and emission combinations. A sorting flow cytometer, often referred to as fluorescence-activated cell sorters, can deflect the stream of cells to isolate single cells that have a specific fluorescence fingerprint that falls within the selected gates (sorting gates). Cells that are not deflected are discarded. Chlorella microalgae strains having a chlorophyll content below 0.5 mg/g DCW were therefore identified by their FI and were deflected into a single tube, creating an enriched pool of genetically unique mutants with similar fluorescence phenotypes. Alternatively, they can be sorted into individual tubes, or wells within a microplate, to isolate single mutant lines.

A positive control was used to calibrate or specify the sorting gates for selecting cells with the desired fluorescent properties. This positive control was wild-type cells which had their chlorophyll extracted using 90% acetone and photobleached for 20-30 minutes, or existing mutant strains that have the desired chlorophyll content. A negative control is also used to calibrate the sorting gates. The negative control was the parent strain of the mutant pool, which has above, at most, 0.5 mg/g DCW chlorophyll. The positive controls had a low FI signal at 495 nm excitation, 695±40 nm emission channel, 561 excitation, 695±40 nm emission channel. The negative control exhibited a high signal in the same channels. Fluorescent properties associated with low chlorophyll, of all cells (within a sampled pool) within the negative control should fall outside the sorting gate, to avoid sorting false positives.

Once the gates were calibrated the mutant pool of cells can be sorted, isolating cell lines with below 0.5 mg/g DCW chlorophyll content. Both the positive and negative control were used to gate to exclude unhealthy or dead cells using forward and side scatter of the 488 nm laser.

The enriched population of mutants with 0.5 mg/g DCW chlorophyll and a CIELAB L* value of greater than about 78 were resorted with narrower range of sorting gates, excluding cells with trace concentrations of pigment and enriching cells with nil pigment, to identify and isolate variant strains with a L*a*b* colour space with an L* value in the range of between 81.0 and 85.0; an a* value in the range of between 1.8 and 3.1; and a b* value in the range of between 16.0 and 19.0. This additional sorting step was achieved using a combination of 495 nm excitation, 670±30 nm emission channel, 561 excitation, 685±15 nm emission channel, in addition to 349 nm excitation, 420±10 nm emission channel, 405 nm excitation, 661±20 nm emission channel, and 445 nm excitation, 650 LP nm emission channel.

Identifying and Isolating Variant Strains of Chlorella Microalgae with an Increased Protein Content and Reduced Starch Content

Isolation of suitable variants may be performed by any means known to the skilled person. The use of a staining or indicator agents, such as iodine, including iodine vapor or solution, preferably iodine vapour, to stain the intracellular starch present within the cell, specifically within the chloroplast, flow cytometry or a combination thereof are preferred.

The identification of a modified strain of Chlorella microalgae comprises sorting or screening the cells by any suitable technique, such as by using flow cytometry. The protein and starch modified strain of Chlorella microalgae may, be selected based upon the degree of staining by iodine vapour, detectable by visual inspection or intensity of starch-iodine fluorescence signal obtained on cell sorting by flow cytometry. The modified strain of Chlorella microalgae may be further selected, based on a desirable pigment or cell wall composition, wherein the desirable pigment or cell wall composition is based upon a relative signal obtained on cell sorting by flow cytometry. The use of flow cytometry provides the advantages of examining thousands of cells per second and in real time and processing quantifiable data over a computer coupled to a flow cytometer. Furthermore, flow cytometry helps in cell counting, cell sorting, determining cell characteristics and function and detecting microorganisms.

Isolated mutant strains of Chlorella microalgae, derived from a chlorophyll deficient parent mutant strain, grown on solid nutrient replete media, were stained in a sealed glass container saturated with iodine vapor; 5 g of iodine granules were placed on the clean lid of a petri dish, located within a wide 1 L glass beaker. The corresponding bottom of the second petri dish, holding the Chlorella colonies, is then positioned above the iodine granules, with colonies facing downwards, and the beaker is sealed with a lid for 1.5 minutes to enable staining. After staining, colonies with higher starch content appear darker and can be selected for resuspension in 20 μL of HSM+3% glucose medium and subsequently spotted onto HSM+3% glucose plates. Alternative chemicals can be bound to intracellular starch or protein, with different spectral properties. Iodine vapor permeates through the cell wall and cell membrane. Polyiodide ions (Iₙ⁻) form a complex with the amylose fraction of starch; this complex has specific light absorption spectra, appearing dark blue. When cells with the wild-type phenotype (i.e. high starch) are stained, a dark blue colour results, corresponding to a high concentration of starch, including amylose. Mutant strains of Chlorella microalgae with a different, lighter or undetectable colour change compared to the parent strain (dark blue) were selected. Strains with mutations in starch biosynthesis, can exhibit higher protein content.

Alternatively, the mutant strains of Chlorella microalgae were stained with iodine solution (2% KI w/v and 1% I₂ w/v) for 1-60 minutes. Samples were washed with phosphate-buffered saline (PBS) or liquid media to remove the excess iodine. Resuspended, stained cells were sorted by flow cytometry, with 488 nm excitation, according to their fluorescence emission shift at 515 nm (or between 500-530 nm) and compared to the non-mutated parent strain of Chlorella microalgae (namely, control). A single suspension of cells was prepared, effectively stained, and allowed to flow through the flow cytometer in a single flow through the light beam for sensing. The laser was used to elicit strong fluorophore fluorescence from intracellular starch-iodine complexes in viable cells. The dye-specific fluorescence signals were analysed by a computer physically connected to the flow cytometer. The population of cells that exhibited strong fluorescence was sorted away from those cells that had null or significantly reduced signal as an enrichment step to enrich for those cells within the total population that had accumulated mutations that knock-down or abolish the starch content signal. This step was applied between 2-7 days post-exposure to mutagen and in liquid culture.

The screening step was repeated up to 5 times in order to isolate cells with a stable genetic trait, rather than strains with an unstable and variable trait due, for instance, to natural phenotypic plasticity (i.e. not due to stable, inherited genetic mutation). Furthermore, both the mutagenesis and screening step can be repeated on the same strain lineage to isolate mutants with still lower starch content, thereby selecting strains with multiple knock-out or knock-down mutations in different genes or associated genetic regulatory elements involved in the starch synthesis or protein synthesis pathway.

The identification of starch deficient strains from chlorophyll-deficient parents (due to stable, inherited genetic traits) was possible under nutrient replete conditions (i.e. without nitrogen deficiency induced chlorosis), demonstrating that this method enables isolation of mutants of Chlorella microalgae that have a genetically-determined, constitutively lower starch content, rather than those which exhibit starch accumulation in response to, for example nutrient stress.

Parent strain selection is important for the strain development process; parent strains of Chlorella microalgae with a desired phenotype are screened for high protein, and high growth rate prior to mutagenesis and screening of starch deficiency (i.e. resulting in higher protein levels). The starch content was verified using the Total Starch Assay Kit Assay Kit™ (K-TSTA; Megazyme, Ireland). Further, the manufacturer's “total starch content of samples containing resistant starch (RTS-NaOH)” procedure was followed.

Selected Chlorella microalgae mutants, characterised by starch deficiency, were further screened to ensure a higher protein content was maintained in various, controlled, growth conditions. Protein quantification was carried out using several methods, including the Biuret, Bradford, BCA, Lowry, Fluorescent, Pierce, Kjeldahl, Dumas methods, and amino acid quantification. The Dumas method is preferred due to its suitability in determining total protein content. This method involves combusting a small, known mass of sample (100 mg-1 g) at high temperatures (800-900° C.) in the presence of oxygen. This combustion process leads to the release of gases, including nitrogen. The nitrogen gas is then separated and quantified using a thermal conductivity detector, providing an estimation of crude protein content. This nitrogen value is converted to a protein value using the established nitrogen to protein (NtP) conversion factor of 6.25 (Nx6.25), as per McCance and Widdowson's “The Composition of Foods” (ISBN 978-1-84973-636-7). This factor is widely accepted for various foods, including microalgae, and it normalises the protein content to make it comparable with other protein sources.

For the Dumas analysis, Chlorella microalgae cultures were grown in glass Erlenmeyer flasks using nutrient-rich media supplemented with glucose. The cultures were maintained in the dark at 28° C. with 120 rpm agitation for 3-7 days until a biomass density of 4-8 g/litre was achieved. Sampling occurred during late exponential growth phase to avoid stress-induced variations. Alternatively, samples were also obtained from cells cultured in controlled batch-fed fermenters, harvested during exponential growth at densities ranging from 4-170 g/l. Prior to Dumas analysis, the samples were washed with distilled water and freeze-dried. This method does not account for variation in non-protein nitrogen.

Alternatively, the protein content was measured by amino acid content. In this method, the dry biomass (50 g) produced by the same method as above is processed by a suitable method to break down the proteins into separate amino acids (hydrolysis). The amino acids were derivatised to aid detection, then separated with ion exchange chromatography, liquid chromatography (LC), high pressure liquid chromatography (HPLC), other similar chromatography, gas chromatography, and detected with UV, fluorescence, pulse amperometry, flame ionisation detection (FID), mass spectrophotometry (MS) or nuclear magnetic resonance (NMR).

Strains with protein contents higher than 50% (Dumas 6.25 NtP) were selected. This method resulted in a systematic, consistent, directed increase in the desired phenotype, i.e. increased relative protein concentration in a chlorophyll-deficient Chlorella.

Chlorella vulgaris:
Genetically Identifying Chlorella vulgaris Using PCR Amplification

Wild-type Chlorella vulgaris 4TC3 microalgae was genetically identified by 18S and ITS2 sequencing as described herein above.

Experimental Protocol for Isolation of Chlorella vulgaris YC03:

Mutant YC03 was isolated by mutating 4TC3/16 and screening via visual plate screening. 4TC3/16 was grown to exponential phase in HSM+1% glucose media in the light, at 25° C. and with 120 rpm agitation. Cells were concentrated to 1×10⁹ cells per ml, in 1 ml of HSM+1% glucose media. A 51 μl aliquot of EMS was added (0.5M final concentration) and the culture was incubated for 2 hours. The cells were washed 3 times and left to recover in HSM+1% glucose media in the dark, at 25° C. and 120 rpm agitation. After 24 hours, the cells were plated on HSM+1% glucose agar plates in aliquots of 5000 cells per plate. Plates were stored in the dark at 28° C. After 3 weeks plates were visually screened, colonies with low levels of chlorophyll, including YC03 were isolated and sub cultured into 25 ml HSM+3% glucose for further validation of chlorophyll and carotenoid content. Cultures were maintained in the same conditions as described above.

YC03 isolated in this manner was yellow in colour, had a chlorophyll content of 0.05 mg/g, and a protein content of 35.95% w/w.

Experimental Protocol for Isolation of Chlorella vulgaris WC03:

Mutant WC03 was isolated by mutating YC03 and screening via visual plate screening. YC03 was grown to exponential phase in FERM media, in the dark at 28° C. and 120 rpm agitation. Cells were concentrated to 1×10⁹ cells per ml, in 1 ml of HSM+1% glucose media. A 10 μl aliquot of MMS was added and the culture was incubated for 1 hours. After quenching the mutagen with the addition of 30% (final concentration 5%) sodium thiosulphate, the cells were washed 3 times and left to recover in HSM+3% glucose media in the dark, at 25° C. and 120 rpm agitation. After 24 hours, the cells were plated on HSM+3% glucose agar plates in aliquots of 5000 cells per plate. After 3 weeks plates were visually screened, colonies with low levels of carotenoid and chlorophyll, including WC03 were isolated and sub cultured into 25 ml HSM+3% glucose for further validation of chlorophyll and carotenoid content. Cultures were maintained in the same conditions as described above.

WC03 isolated in this manner was white in colour, had a chlorophyll content of 0.001 mg/g, and a protein content of 34.5% w/w.

Experimental Protocol for Isolation of Chlorella vulgaris YC27:

Mutant YC27 was isolated by mutating 4TC3/16 and screening via flow cytometry.

Herein follows a description of the preferred method for isolating chlorophyll deficient Chlorella microalgae strains using flow cytometry cell sorting. In this example, the chlorophyll-deficient Chlorella is Chlorella vulgaris mutant strain YC27 (parent strain of WCLS06). However, the described method is suitable to isolate chlorophyll deficient mutants of Chlorella microalgae in general.

Exponential phase wild type (4TC3/16, chlorophyll replete) cells were concentrated to 1×10⁹ cells per ml in HSM+3% glucose media. An aliquot of methyl methanesulphonate (MMS) was added to the cell solution at a final concentration of 0.12M (10 μl per 1 ml). The cell mixture was incubated for 1 hour in the dark. A 30% sodium thiosulphate solution was added to the cell mixture to a final concentration of 5% sodium thiosulphate. The culture was incubated for 10 minutes. The cells were washed 3 times in 25 ml of HSM+1% glucose. The cells were resuspended in 25 ml HSM+3% glucose and left to recover for 144 hours in the dark, at 28° C. and 120 rpm agitation.

After a period of recovery the cells were sub-cultured into fresh media, further incubated, and sampled during mid-exponential phase growth (5×10⁶ cells ml⁻¹ to 5×10⁷). 100,000 cells were initially analysed with a BD FACSAria Fusion (Becton Dickinson, USA) or Bigfoot Spectral Cell Sorter (Thermo Fisher Scientific, USA) in order to detect their fluorescence properties. In addition to the mutant pool, wild type cells and chlorophyll deficient mutants (isolated by alternative methods or previous flow cytometry isolation) were analysed. These single strain cells were used as controls to design gates to sort populations.

First, using the forward and side scatter (FSC and SSC parameters) of the excitation laser, healthy single cells were gated, excluding cell debris and clumped or dividing cells. The chlorophyll content of cells was analysed by measuring the emission fluorescence intensity at 695±40 (or 670±30) after excitation from 488 nm and 561 nm lasers. Gates were designed based on the fluorescence properties of the control cells. Gates were drawn to instruct the flow cytometry sorter to isolate cells that have fluorescence properties within the range of fluorescent intensities which match the chlorophyll deficient control population, excluding to the wild type chlorophyll replete control cell population.

Using live data and prepared gates, cells with the desired chlorophyll deficient fluorescent properties were sorted away from a stream of a mixed mutant population into a tube or microplate well. The nozzle tip size used for sorting was 100 μm. The sorted cells were sorted into 100 μl HSM+3% glucose media with 300 μg/ml carbenicillin and 85 μg/ml cefotaxime. After 2 weeks of growth, single cells multiplied into large populations. The chlorophyll content was screened by visual colour. False positive mutant populations were discarded, chlorophyll deficient mutant populations were scaled up to a larger cell culture and validated by chlorophyll analysis.

4TC3/16 was grown to exponential phase in FERM media in the dark, at 28° C. and with 120 rpm agitation. Cells were concentrated to 1×10⁹ cells per ml, in 5 ml of HSM+1% glucose media. A 50 μl aliquot of MMS was added and the culture was incubated for 1 hour. After quenching the mutagenesis with the addition of 30% (final concentration 5%) sodium thiosulphate, the cells were washed 3 times and left to recover in HSM+3% glucose media for 6 days and sub-cultured. After 4 days, when the culture had reached mid-exponential phase growth, cells were sorted by flow cytometry sorting (florescence activated cell sorting, FACS), using 488 nm and 561 nm lasers and 670±30 nm emission as previously described herein. Cells with low florescence intensity matching chlorophyll deficient control cell population were gated away from the main population and sorting into 96-well microplates, with 100 μl HSM+3% glucose media with 300 μg/ml carbenicillin and 85 μg/ml cefotaxime. The cells were incubated in the same conditions described above. After 2 weeks chlorophyll deficient colonies, including YC27 were sub cultured into 25 ml HSM+3% glucose media for further validation.

YC27 isolated in this manner was yellow in colour, had a chlorophyll content of 0.15 mg/g, and a protein content of 45.9% w/w.

Experimental Protocol for Isolation of Chlorella vulgaris WC12:

Mutant WC12 was isolated by mutating YC27 as previously described herein and screening via visual plate screening. YC27 was grown to exponential phase in FERM media, in the dark at 28° C. and 120 rpm agitation. Cells were concentrated to 1×10⁹ cells per ml, in 1 ml of HSM+1% glucose media. A 51 μl aliquot of EMS was added (0.5M final concentration) and the culture was incubated for 2 hours. After quenching the mutagenesis with the addition of 30% (final concentration 5%) sodium thiosulphate, the cells were washed 3 times and left to recover in HSM+3% glucose media. After 24 hours, the cells were plated on HSM+3% glucose agar plates. An aliquot of 5000 cells per plated on each plate. After 3 weeks plates were visually screened, colonies with low levels of carotenoid, including WC12 were isolated and sub cultured into 25 ml HSM+3% glucose for further validation of chlorophyll and carotenoid content. Cultures were incubated in the same conditions as described above.

WC12 isolated in this manner was white in colour, had a chlorophyll content of 0.04 mg/g, and a protein content of 45.2% w/w.

Experimental Protocol for Isolation of Chlorella vulgaris WCLS04, WCLS05 and WCLS06:

Mutants WCLS04, WCLOS05 and WCLOS06 were isolated by mutating WC12 and screening via starch-stained plate screening, as follows. WC12 was grown to exponential phase in FERM media, in the dark at 28° C. and 120 rpm agitation. Cells were concentrated to 1×10⁹ cells per ml, in 1 ml of HSM+1% glucose media. A 51 μl aliquot of EMS was added (0.5M final concentration) and the culture was incubated for 1 hours. After quenching the mutagenesis with the addition of 30% (final concentration 5%) sodium thiosulphate, the cells were washed 3 times and left to recover in HSM+3% glucose media. After 24 hours, the cells were plated on HSM+3% glucose agar plates. An aliquot of 5000 cells per plated on each plate. After 4 weeks plates were stained with iodine vapour for 1.5 minutes (using the iodine granule method; as described herein above). Colonies with low levels of stain compared to non-mutated controls and the majority of the mutant population were isolated and restreaked onto HSM+3% glucose agar plates. After 2 weeks, the iodine staining was repeated to ensure consistent low staining. Single colonies were isolated and scaled up for starch and protein analysis. From these colonies WCLS04, WCLS05, WCLS06 were isolated.

WCLS04 isolated in this manner was white in colour, had a chlorophyll content of <0.03 mg/g, and a protein content of 56.2% w/w.

WCLS05 isolated in this manner was white in colour, had a chlorophyll content of <0.03 mg/g, and a protein content of 54.1% w/w.

WCLS06 isolated in this manner was white in colour, had a chlorophyll content of <0.03 mg/g, and a protein content of 58.7% w/w.

Genomic Sequence Analysis of Chlorella vulgaris WC03 (Examples 1, 2 and 3), WC12 (Example 4), WCLS04 (Example 5) and WCLS06 (Example 6): There follows a description of the preferred method for genome sequencing and annotation of Chlorella microalgae. Optionally, the Chlorella microalgae is Chlorella vulgaris mutant strain WC03. However, the described method is suitable to identify genetic mutations in Chlorella microalgae in general.

Genome sequencing of Chlorella vulgaris WC03 was performed using Illumina sequencing, resulting in a final genome assembly of 38.1 Mbp with an average GC content of 61.5%. A total of 10,542 genes were predicted and annotated via InterProScan and KEGG, with variant analysis identifying 25 mutations in WC03 compared to Chlorella vulgaris 4TC3, consisting of 11 SNPs and 14 INDELs.

An axenic culture of Chlorella vulgaris WC03 was cultivated from a thawed cryostock in FERM complete media at 28° C. under heterotrophic conditions with shaking at 130 rpm. Cells were harvested after seven days by centrifugation at 13300×g for 5 minutes. DNA was extracted from Chlorella vulgaris WC03 biomass by mechanical bead-beating and TRIzol™ reagent. Extracted DNA was quantified via Nanodrop™ and DNA integrity was assessed by gel electrophoresis on a 1% agarose gel. Library preparation, including DNA fragmentation, adapter ligation, amplification and size selection was performed by Eurofins Genomics using proprietary methods. The quality of the final library was assessed by determination of size distribution and quantification, prior to sequencing on the Illumina NovaSeq 6000 platform using 2×150 sequence mode. Quality filtering of genetic data was performed by Eurofins Genomics using Illumina CASAVA software (95% of the bases with a quality of PHRED score 28 or better, no adapter trimming) prior to the reporting of raw reads. Adaptor sequences were removed with Trimmomatic (v0.38.0, Bolger et al., 2014; DOI: 10.1093/bioinformatics/btu170) and quality was checked after trimming using FastQC (v0.11.8, Andrews, 2010; online). Reference-based contig assembly was performed using SPAdes (v 3.12.0, Bankevich et al., 2012; DOI: 10.1089/cmb.2012.0021) with careful correction and automatic k-mer value detection, with the complete genome sequence of Chlorella vulgaris 4TC3 used as the reference. Coverage was assessed via Qualimap 2 (v 2.2.2, Okonechnikov et al., 2015; DOI: 10.1093/bioinformatics/btv566) and determined to be approximately 186× coverage with 99.7% of reads mapping to the 4TC3 reference genome. The statistics of the final assembly are reported in Table 1.

TABLE 1
Features of the Chlorella vulgaris 4TC3 and WC03 genome assemblies.
4TC3 is a complete genome sequence, WC03 is a draft genome sequence;
accounting for the differencwqe in total length.

| | Chlorella vulgaris | |
|---|---|---|
| | 4TC3 (WGS contigs | |
| | are sequences | |
| | 29-62 (SEQ ID | Chlorella vulgaris |
| | NO: 29-62)) | WC03 |
| Total length (bp) | 40,421,215 | 38,068,186 |
| Number of contigs | 32 | 3,108 |
| N50 (bp) | 2,556,997 | 194,580 |
| GC (%) | 61.5 | 61.5 |

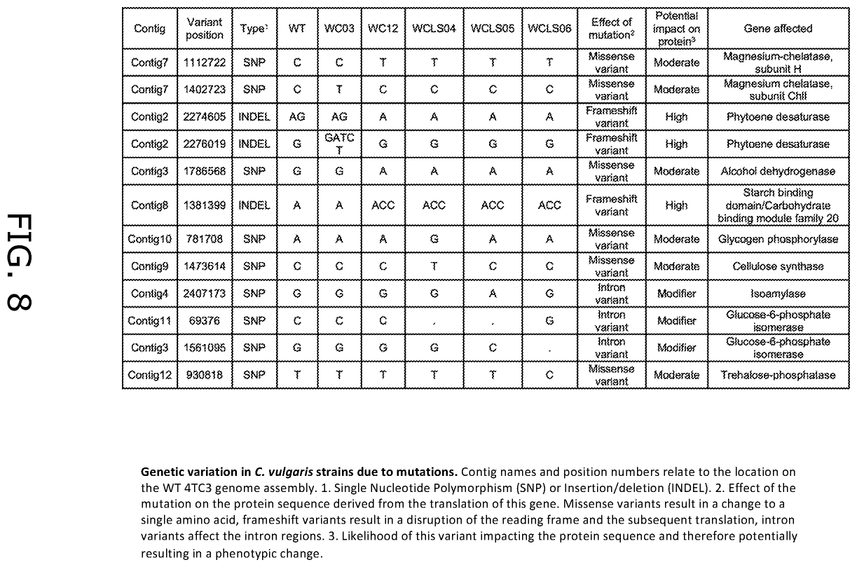

The completeness of the genome assembly was further assessed by the single copy orthologs (BUSCO, v 5.2.2, Manni et al., 2021; DOI: 10.1093/molbev/msab199) with WC03 being 95.7% complete and 1.4% partial genes of the 1519 belonging to the Chlorophyta dataset identified in WC03. Gene prediction was carried out by alignment of gene-models from Chlorella vulgaris 4TC3 with the WC03 genome assembly using Exonerate included in MAKER (v 2.31.11, Cantarel et al., 2008; DOI: 10.1101/gr.6743907). The ab initio gene predictor Augustus (v 3.4.0, Stanke et al., 2004; DOI: 10.1093/nar/gkh379) was trained and a second round of gene prediction in the soft-masked genome was performed using the MAKER pipeline combining the homology-based predictions and ab initio gene prediction, with repeats identified via RepeatMasker (v 4.0.9, Smit et al., 2013; online) and Dfam (v 3.5, Storer et al., 2021; DOI: 10.1186/s13100-020-00230-y). A total of 10,542 genes were identified with BUSCO analysis (v 5.2.2, Manni et al., 2021) identifying 95.1% complete and 2.4% partial genes of the 1519 belonging to the Chlorophyta dataset. For gene function annotation, protein-coding genes were translated into amino acid sequences via the MAKER-P pipeline and annotated using InterProScan (v 5.0.0, Blum et al., 2021; DOI: 10.1093/nar/gkaa977) against the TIGRFAM, Panther and PfamA databases. Proteins were also mapped against the KEGG database (v 101.0, Kanehisa et al., 2022; DOI: 10.1002/pro.4172). A total of 1571 genes were assigned to a metabolic pathway via KEGG analysis. To identify genetic variations in Chlorella vulgaris WC03, the assembled, annotated genome sequence was aligned to the reference genome of 4TC3 using BWA-MEM (v 0.7.17.2, Li and Durbin, 2009; DOI: 10.1093/bioinformatics/btp324). Sorting and dereplication were performed using Picard (v 2.26.10, Broad Institute, 2019) prior to base recalibration and variant calling using GATK BQSR and Haplotype Caller (v 4.1.3.0, Poplin et al., 2017; DOI: 10.1101/201178) with ploidy set to 1. Variant filtering was performed using GATK Select Variants and Variant Filtration tools. All variants were filtered with the Quality by Depth filter of <2.0 and Quality <30.0. Additional filtering was performed on SNPs with FisherStrand >60.0 and RMSMappingQuality <40.0, and on INDELs with FisherStrand >200.0. SnpEff (v4.3, Cingolani et al., 2012; DOI: 10.4161/fly.19695) was used to annotate and predict the effect of the variants on gene function. Functional information for the identified genes was obtained by InterProScan (v 5.0.0, Blum et al., 2021). A total of 25 mutations were identified, comprising 11 SNPs and 14 INDELs. Genetic variations in Chlorella vulgaris strains due to mutations identified herein are summarised in FIG. 8. The likelihood of a variant impacting the protein sequence and ascribed function of the protein and, therefore, potentially resulting in a phenotypic change is summarised in Table 2.

TABLE 2
Putative variant impact as identified
via SnpEFF (Cingolani et al., 2012)

| Impact | Meaning | Example |
|---|---|---|
| High | The variant is assumed to | Stop gained |
| | have high (disruptive) impact | Frameshift variant |
| | in the protein, probably | |
| | causing protein truncation, | |
| | loss of function or triggering | |
| | nonsense mediated decay. | |
| Moderate | A non-disruptive variant that | Missense variant |
| | might change protein | In-frame deletion |
| | effectiveness. | |
| Low | Assumed to be mostly | Synonymous variant |
| | harmless or unlikely to change | |
| | protein behaviour. | |
| Modifier | Usually non-coding variants or | Exon variant |
| | variants affecting non-coding | Downstream gene variant |
| | genes, where predictions are | |
| | difficult or there is no evidence | |
| | of impact. | |

Where there is reference to specific mutations, the positions are described in reference to the Wild Type genome sequence. To identify these positions the mutant genome sequence is mapped against the relevant reference genome. The term “Contigs” refer to specific contigs of the reference genome (4TC3 for C. vulgaris, UTEX1230 for C. sorokiniana) and the “variant position” is specific to this contig (position numbers restart from 1 at each new contig). For example, a variant in C. vulgaris at Contig 11 position 69376 would be found in the variant sequences that align to Contig 11 of 4TC3, at the position located 69376 bases from the start of the sequence.

Genetic Description of Chlorella vulgaris Strains YC03, WC03, YC27, WC12, WCLS04, WCLS05 and WCLS06

There follows a genetic description of Chlorella vulgaris WC03 and various Chlorella vulgaris strains that form embodiments of the invention. It will be appreciated that the skilled person could use this information to reproduce such strains and, thereby, various embodiments of the invention without undue experimentation by using direct gene-editing methods, (in addition to the mutagenesis methods described herein). Such suitable gene editing tools or methods that would be known to the person skilled in the art include, but are not limited to: genetic recombination, zinc finger nucleases, transcription activator-like effector nucleases (TALENS), CRISPR-Cas9 gene editing, base editing (e.g. using dCas9), prime editing (e.g. using pegRNA) or Programmable Addition via Site-specific Targeting Elements (PASTE).

YC03 is characterised by mutations in the gene encoding magnesium chelatase, subunit I (ChII). It exhibits a reduction in chlorophyll in comparison to the WT strain 4TC3.

WC03 is characterised by mutations in genes encoding magnesium chelatase, subunit I (ChII; Sequence 2 (SEQ ID NO: 2)) and phytoene desaturase (Sequence 4 (SEQ ID NO: 4), EC: 1.3.5.5). It exhibits a reduction in chlorophyll in comparison to the WT strain 4TC3.

YC27 is characterised by mutations in the gene encoding magnesium-chelatase, subunit H (ChIH). It exhibits a reduction in chlorophyll in comparison to the WT strain 4TC3.

WC12 is characterised by mutations in the genes encoding magnesium-chelatase, subunit H (ChIH; Sequence 7 (SEQ ID NO: 7)), phytoene desaturase (Sequence 5 (SEQ ID NO: 5), EC: 1.3.5.5), alcohol dehydrogenase (Sequence 9 (SEQ ID NO: 9)), and starch binding domain (Sequence 11 (SEQ ID NO: 11)). It exhibits a reduction in chlorophyll in comparison to the WT strain 4TC3. It also exhibits an increase in protein content and decrease in starch content compared to WT strain, 4TC3 and WC03.

WCLS04 is characterised by mutations in the genes encoding magnesium-chelatase, subunit H (Sequence 7 (SEQ ID NO: 7)), phytoene desaturase (Sequence 5 (SEQ ID NO: 5), EC: 1.3.5.5), alcohol dehydrogenase (Sequence 9 (SEQ ID NO: 9)), starch binding domain gene (Sequence 11 (SEQ ID NO: 11)), glycogen phosphorylase (Sequence 13 (SEQ ID NO: 13), EC: 2.4.1.1) and cellulose synthase (UDP-forming) (Sequence 15 (SEQ ID NO: 15), EC: 2.4.1.12). It exhibits a reduction in chlorophyll in comparison to the WT strain 4TC3. It also exhibits an increase in protein and decrease in starch compared to 4TC3, and WC12.

WCLS05 is characterised by mutations in the genes encoding magnesium-chelatase, subunit H (Sequence 7 (SEQ ID NO: 7)), phytoene desaturase (Sequence 5 (SEQ ID NO: 5), EC: 1.3.5.5), alcohol dehydrogenase (Sequence 9 (SEQ ID NO: 9)), starch binding domain gene (Sequence 11 (SEQ ID NO: 11)), isoamylase (Sequence 17 (SEQ ID NO: 17), EC: 3.2.1.68) and glucose-6-phosphate isomerase (Sequence 19 (SEQ ID NO: 19), EC: 5.3.1). It exhibits a reduction in chlorophyll in comparison to the WT strain 4TC3. It also exhibits an increase in protein and decrease in starch compared to 4TC3 and WC12.

WCLS06 (whole genome sequence 63 (SEQ ID NO: 63)) is characterised by mutations in the genes encoding magnesium-chelatase, subunit H (Sequence 7 (SEQ ID NO: 7)), phytoene desaturase (Sequence 5 (SEQ ID NO: 5), EC: 1.3.5.5), alcohol dehydrogenase (Sequence 9 (SEQ ID NO: 9)), starch binding domain gene (Sequence 11 (SEQ ID NO: 11)), trehalose 6-phosphate synthase (Sequence 23 (SEQ ID NO: 23), EC 3.1.3.12) and glucose-6-phosphate isomerase (Sequence 21 (SEQ ID NO: 21), EC: 5.3.1.9). It also exhibits an increase in protein and decrease in starch compared to 4TC3 and WC12.

Chlorella sorokiniana:
Genetically Identifying Chlorella sorokiniana Using PCR Amplification

Wild-type Chlorella sorokiniana UTEX 1230 was obtained from UTEX Culture Collection of Algae at UT-Austin, Texas, USA. It was genetically verified in-house by 18S and ITS2 sequencing as described above.

Experimental Protocol for Isolation of Chlorella sorokiniana CS04 (YSK04):

Strain CS04 was isolated by mutating Chlorella sorokiniana UTEX1230 and screening via visual plate screening. UTEX1230 was grown to exponential phase in FERM media. The cell concentration was adjusted to 0.5×10⁹ cells per ml in a 0.5 mL volume of their respective growth media. Mutagenesis was performed using 0.5M EMS and incubating for 1 hour. After quenching the mutagenesis with the addition of 30% (final concentration 5%) sodium thiosulphate, the cells were washed 3 times and left to recover in FERM media for 2 days.

After the recovery period, the cells were plated on HSM media enriched with 1% glucose and vitamins (HSM1GV) agar plates in aliquots of 500 cells per plate and incubated at 25° C. in the dark. After 1-2 weeks plates were visually screened, and colonies with a yellow phenotype, including CS04, were isolated. From these plates, yellow UTEX1230 mutants were isolated, initially cultured in 500 μL snap cap tubes, and later transferred to 20 mL FERM media in 50 mL flasks for further growth. Over a period of 2-3 weeks, these mutants predominantly maintained a yellow coloration. One particular yellow mutant was identified and named CS04.

CS04 isolated in this manner was yellow in colour, had a chlorophyll content of 0.47 mg/g, and a protein content of 33.8% w/w.

Experimental Protocol for Isolation of Chlorella sorokiniana CS10 (WSK04) and CS11 (WSK05), CS12 (WSK06):

Mutants CS10, CS11 and CS12 were isolated by mutating CS04 and screening via starch-stained plate screening, as follows. CS04 was grown to exponential phase in FERM media, in the dark at 28° C. and 120 rpm agitation. Cells were concentrated to 1×10⁹ cells per ml, in 1 ml of HSM+1% glucose media. A 51 μl aliquot of EMS was added (0.5M final concentration) and the culture was incubated for 1 hours. After quenching the mutagenesis with the addition of 30% (final concentration 5%) sodium thiosulphate, the cells were washed 3 times and left to recover in HSM+3% glucose media. After 24 hours, the cells were plated on HSM+3% glucose agar plates. An aliquot of 5000 cells per plated on each plate. After 4 weeks plates were stained with iodine vapour for 1.5 minutes (using the iodine granule method; as described herein above). Colonies with low levels of stain compared to non-mutated controls and the majority of the mutant population were isolated and restreaked onto HSM+3% glucose agar plates. After 2 weeks, the iodine staining was repeated to ensure consistent low staining. Single colonies were isolated and scaled up for starch and protein analysis. From these colonies CS10, CS11 and CS12 was isolated.

CS10 isolated in this manner was white in colour, had a chlorophyll content of <0.07 mg/g, and a protein content of 51.32% w/w.

CS11 isolated in this manner was white in colour, had a chlorophyll content of <0.07 mg/g, and a protein content of 50.10% w/w.

CS12 isolated in this manner was white in colour, had a chlorophyll content of <0.07 mg/g, and a protein content of 51.67% w/w.

Experimental Protocol for Isolation of Chlorella sorokiniana CS107:

Strain CS107 was isolated by mutating CS04 and screening via visual plate screening. CS04 was grown to exponential phase in FERM media, in the dark at 28° C. and 120 rpm agitation. Cells were concentrated to 1×10⁹ cells per mL, in FERM media and incubated in the presence of 0.5M EMS for 1 hour. After quenching the mutagen with the addition of 30% (final concentration 5%) sodium thiosulphate, the cells were washed 3 times and left to recover in ¼ FERM media in the dark, at 25° C. and 120 rpm agitation. After 24 hours, the cells were plated on FERM agar plates in aliquots of 1000 cells per plate and incubated at 25° C. in the dark. After 2 weeks plates were visually screened, and colonies with a white phenotype, including CS107, were isolated and sub cultured into 25 mL FERM for further validation of chlorophyll and carotenoid content. Cultures were maintained in the same conditions as described above.

CS107 isolated in this manner was white in colour, had a chlorophyll content of 0.074 mg/g, and a protein content of 43% w/w.

Experimental Protocol for Isolation of Chlorella sorokiniana CS172:

Strain CS172 was isolated by mutating CS107 and screening via visual plate screening. CS107 was grown to exponential phase in FERM media, in the dark at 28° C. and 120 rpm agitation. Cells were concentrated to 1×10⁹ cells per mL, in FERM media and incubated in the presence of 0.5M EMS for 1 hour. After quenching the mutagen with the addition of 30% (final concentration 5%) sodium thiosulphate, the cells were washed 3 times and left to recover in ¼ FERM media in the dark, at 32° C. and 120 rpm agitation. After 24 hours, the cells were plated on FERM agar plates in aliquots of 700 cells per plate and incubated at 32° C. in the presence of 20% CO₂, in the dark. After 3 weeks plates were visually screened and colonies with a white phenotype, including CS172, were isolated and sub cultured into 25 mL FERM for further validation of chlorophyll and carotenoid content. Cultures were maintained in the same conditions as described above.

CS172 isolated in this manner was white in colour, had a chlorophyll content of 0.054 mg/g, and a protein content of 31.9% w/w.

Experimental Protocol for Isolation of Chlorella sorokiniana CS174:

Strain CS174 was isolated by mutating CS172 and screening via starch staining plate screening as previously described herein. CS172 was grown to exponential phase in FERM media, in the dark at 28° C. and 120 rpm agitation. Cells were concentrated to 1×10⁹ cells per ml, in FERM media and incubated in the presence of 0.5M EMS for 1 hour. After quenching the mutagen with the addition of 30% (final concentration 5%) sodium thiosulphate, the cells were washed 3 times and left to recover in ¼ FERM media in the dark, at 32° C. and 120 rpm agitation. After 24 hours, the cells were plated on FERM agar plates in aliquots of 1000 cells per plate and incubated at 28° C. in the dark. After 1 week, plates were visually screened and colonies with a white phenotype, including CS174, were isolated and sub cultured into snap cap tubes containing 10 mL ¼ FERM. Colonies were incubated in the dark, at 32° C. and 120 rpm agitation for 72 hours. 1 OD (750 nm) of cells from each colony was resuspended and then replica-plated (in 100 μL spots) onto FERM agar plates. Spots were directly stained with iodine to identify mutant strains exhibiting “low starch” phenotypes; such strains were isolated and scaled up for starch and protein analysis. One such strain was CS174.

CS174 isolated in this manner was white in colour, had a chlorophyll content of 0.052 mg/g, and a protein content of 59% w/w.

Experimental Protocol for Isolation of Chlorella sorokiniana CS73:

Strain CS73 was isolated by mutating Chlorella sorokiniana UTEX1230 and screening via flow cytometry. UTEX1230 was grown to exponential phase in FERM media. The cell concentration was adjusted to 1×10⁹ cells per mL in a 1 mL volume of their respective growth media.

Mutagenesis was performed using 0.5M EMS and incubating for 1 hour. After quenching the mutagenesis with the addition of 30% (final concentration 5%) sodium thiosulphate, the cells were washed 3 times and left to recover in HSM1GV for 3 days and sub-cultured. After multiple subcultures, 13 days, when the culture had reached mid-exponential phase growth, cells were sorted by flow cytometry sorting florescence activated cell sorting (FACS), using 488 nm and 561 nm lasers and 670±30 nm emission as previously described herein.

Sorting gates were set using the distinction in autofluorescence between wild type and mutant yellow control cells, allowing for precise gating of chlorophyll-deficient mutants. New mutant yellow cells of UTEX1230 were sorted into HSM1GV. These cells were then incubated under the same conditions described above. After a recovery period, the cells were plated on HSM+1% glucose media plates. From these plates, yellow UTEX1230 mutants were isolated, initially cultured in 500 μL snap cap tubes, and later transferred to 20 mL FERM media in 50 mL flasks for further growth. Over a period of 2-3 weeks, these mutants predominantly maintained a yellow coloration. One particular yellow mutant was identified and named CS73.

Experimental Protocol Isolation of Chlorella sorokiniana CS120:

Strain CS120 was isolated by mutating CS73 and screening via visual plate screening. CS73 was grown to exponential phase in FERM media, in the dark at 28° C. and 120 rpm agitation. Cells were concentrated to 1×10⁹ cells per mL, in 1 mL of HSM1GV and incubated in the presence of 0.5M EMS for 1 hour. After quenching the mutagen with the addition of 30% (final concentration 5%) sodium thiosulphate, the cells were washed 3 times and left to recover in HSM1GV in the dark, at 25° C. and 120 rpm agitation. After 24 hours, the cells were plated on HSM1GV agar plates in aliquots of 1000 cells per plate and incubated at 25° C. in the dark. After 3 weeks plates were visually screened and colonies with a white phenotype, including CS120, were isolated and sub cultured into 25 mL HSM1GV for further validation of chlorophyll and carotenoid content. Cultures were maintained in the same conditions as described above.

Genomic Sequence Analysis of Chlorella sorokiniana Strains

There follows a description of the preferred method for genome sequencing and annotation of Chlorella microalgae. Genome sequencing of Chlorella sorokiniana UTEX 1230 was performed by (Hovde et al., 2018; DOI: 10.1016/j.algal.2018.09.012) using Illumina and PacBio sequencing, resulting in a final genome assembly of 58.5 Mbp with an average GC content of 63.8%. The genome sequence of C. sorokiniana UTEX 1230 is publicly available to download from the National Center for Biotechnology Information database under bioproject PRJNA422912, genome assembly ASM313072v1.

The statistics of the final assembly are reported in Table 6.

TABLE 6
Features of the C. vulgaris 4TC3 and
C. sorokiniana UTEX 1230 genome assemblies.

| | C. vulgaris 4TC3 | |
|---|---|---|
| | (WGS contigs are sequences | C. sorokiniana |
| | 29-62 (SEQ ID NO: 29-62)) | UTEX 1230 |
| Total length | 40.4 | 58.5 Mb |
| (Mbp) | | |
| Number of | 32 | 20 |
| contigs | | |
| N50 (Mbp) | 2.56 | 3.82 |
| GC (%) | 61.5 | 63.8 |

Gene prediction was carried out by the inventors via alignment of gene-models from C. sorokiniana UTEX 1230 (Blake et al., 2018; DOI: 10.1016/j.algal.2018.09.012) using Exonerate included in MAKER (v 2.31.11, Cantarel et al., 2008; DOI: 10.1101/gr.6743907).

The ab initio gene predictors Augustus (v 3.4.0, Stanke et al., 2004; DOI: 10.1093/nar/gkh379) and Snap (v 2013_11_29, http://korflab.ucdavis.edu/software.html) were trained and a second round of gene prediction in the soft-masked genome was performed using the MAKER pipeline combining the homology-based predictions and ab initio gene prediction, with repeats identified via RepeatMasker (v 4.0.9, Smit et al., 2013; online) and Dfam (v 3.5, Storer et al., 2021; DOI: 10.1186/s13100-020-00230-y). A total of 13,021 genes were identified with BUSCO analysis (v 5.2.2, Manni et al., 2021) identifying 96% complete and 1.9% partial genes of the 1519 belonging to the Chlorophyta dataset. For gene function annotation, protein-coding genes were translated into amino acid sequences via the MAKER-P pipeline and annotated using InterProScan (v 5.0.0, Blum et al., 2021; DOI: 10.1093/nar/gkaa977) against the TIGRFAM, Panther and PfamA databases. Proteins were also mapped against the KEGG database (v 101.0, Kanehisa et al., 2022; DOI: 10.1002/pro.4172). A total of 1822 genes were assigned to a metabolic pathway via KEGG analysis.

There follows a description of the preferred method to identify genetic variations in Chlorella sorokiniana mutant strains by genome sequencing and annotation. Optionally, the mutant strain of Chlorella sorokiniana microalgae is Chlorella sorokiniana CS172. However, the described method is suitable to identify genetic mutations in Chlorella microalgae in general.

Genome sequencing of Chlorella sorokiniana CS172 was performed using Nanopore sequencing, resulting in a final genome assembly of 57.4 Mbp with an average GC content of 63.85%.

An axenic culture of C. sorokiniana CS172 was cultivated from a thawed cryostock in FERM complete media at 28° C. under heterotrophic conditions with shaking at 130 rpm. Cells were harvested after four days by centrifugation at 13300×g for 5 minutes. DNA was extracted from C. sorokiniana CS172 biomass using the Quick-DNA Plant/Seed Miniprep Kit (Zymo).

Extracted DNA was quantified via Nanodrop® and DNA integrity was assessed by gel electrophoresis on a 1% agarose gel. Library preparation was performed using the Ligation Sequencing Kit V14 (SQK-LSK114, Oxford Nanopore) according to the manufacturer's instructions and sequenced on the MinION using a R10.4.1 flow cell. Raw fast5 files were basecalled using Guppy (v 6.5.7, 400 bps) and sequences with a Q score >9 and longer than 200 bp were used for analysis. Assembly was performed using Flye (v 2.9.1, Lin et al., 2016; DOI: 10.1073/pnas. 1604560113) with one round of polishing. The statistics of the final assembly are reported in Table 7.

TABLE 7
Features of the C. sorokiniana UTEX1230
and CS172 genome assemblies.

| | | C. sorokiniana |
|---|---|---|
| | C. sorokiniana | CS172 (WGS sequence 64 |
| | UTEX1230 | (SEQ ID NO: 64) |
| Total length (Mbp) | 58.5 | 57.4 |
| Number of contigs | 20 | 129 |
| N50 (Mbp) | 3.82 | 1.0 |
| GC (%) | 63.80 | 63.85 |

The completeness of the genome assembly was further assessed by the single copy orthologs (BUSCO, v 5.2.2, Manni et al., 2021; DOI: 10.1093/molbev/msab199) with CS172 containing 99.2% complete and 0.3% partial genes of the 1519 belonging to the Chlorophyta dataset.

Sequences were mapped against the published UTEX 1230 genome using Minimap2 (v 2.26, -ax map-ont-L), read groups added using Picard (v 2.26.10), reads sorted and indexed using samtools (v 1.18) and variants called using Medaka (v 1.7.2, model r1041_e82_400bps_fast_g615). Variants were filtered using SnpSift (v 4.3, QUAL >30, DP >2, MQ>40) and SnpEff (v4.3, Cingolani et al., 2012) was used to annotate and predict the effect of the variants on gene function. Functional information for the identified genes was obtained by InterProScan (v 5.0.0, Blum et al., 2021).

Where there is reference to specific mutations, the positions are described in reference to the Wild Type genome sequence. To identify these positions the mutant genome sequence is mapped against the relevant reference genome. The term “Contigs” refer to specific contigs of the reference genome (4TC3 for C. vulgaris, UTEX1230 for C. sorokiniana) and the “variant position” is specific to this contig (position numbers restart from 1 at each new contig). For example, a variant in C. vulgaris at Contig 11 position 69376 would be found in the variant sequences that align to Contig 11 of 4TC3, at the position located 69376 bases from the start of the sequence.

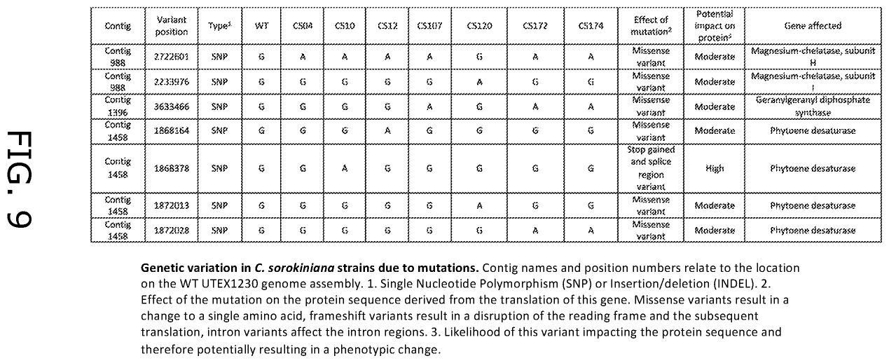

Genetic variations in Chlorella sorokiniana strains due to mutations identified herein are summarised in FIG. 9. The likelihood of a variant impacting the protein sequence and ascribed function of the protein and, therefore, potentially resulting in a phenotypic change is summarised in Table 8.

TABLE 8
Putative variant impact as identified
via SnpEFF (Cingolani et al., 2012)

| Impact | Meaning | Example |
|---|---|---|
| High | The variant is assumed to have | Stop gained |
| | high (disruptive) impact in the | Frameshift variant |
| | protein, probably causing | |
| | protein truncation, loss of | |
| | function or triggering nonsense | |
| | mediated decay. | |
| Moderate | A non-disruptive variant that | Missense variant |
| | might change protein | In-frame deletion |
| | effectiveness. | |
| Low | Assumed to be mostly | Synonymous variant |
| | harmless or unlikely to change | |
| | protein behaviour. | |
| Modifier | Usually non-coding variants or | Exon variant |
| | variants affecting non-coding | Downstream gene variant |
| | genes, where predictions are | |
| | difficult or there is no evidence | |
| | of impact. | |

Genetic Description of Chlorella sorokiniana Strains CS04, CS107, CS172 and CS174

There follows a genetic description of Chlorella sorokiniana CS172, CS174, and various Chlorella sorokiniana strains that form embodiments of the invention. It will be appreciated that the skilled person could use this information to reproduce such strains and thereby, various embodiments of the invention without undue experimentation, by using direct gene-editing methods, (in addition to the mutagenesis methods described herein). Such suitable gene editing tools or methods that would be known to the person skilled in the art include, but are not limited to: genetic recombination, zinc finger nucleases, transcription activator-like effector nucleases (TALENS), CRISPR-Cas9 gene editing, base editing (e.g. using dCas9), prime editing (e.g. using pegRNA) or Programmable Addition via Site-specific Targeting Elements (PASTE).

Geranylgeranyl diphosphate synthase (EC 2.5.1.29) is an enzyme required to the synthesis of Geranylgeranyl diphosphate (GGPP), which is the precursor for the biosynthesis of carotenoids and chlorophylls. Phytoene desaturase (EC 1.3.5.5) is an enzyme essential to the carotenoid biosynthesis pathway and controls the conversion phytoene into lycopene. Magnesium chelatase (EC 6.6.1.1) is an enzyme that catalyses the first committed step of the chlorophyll synthesis pathway; being the insertion of Mg²⁺ into protoporphyrin IX. Magnesium chelatase is a highly-conserved enzyme composed of three subunits: ChII, ChID, and ChIH. The subunits are postulated to have distinct roles in forming the catalytically-active holoenzyme that, ultimately, performs the magnesium chelation reaction; broadly, ChII and ChID are thought to form an ATP-associated complex, while ChIH binds to the magnesium ion, leading to the formation of the active Mg chelatase holoenzyme (Xhang et al. 2018; DOI: 10.3389/fpls.2018.00720).

CS04 is characterised by mutations in the genes encoding magnesium-chelatase, subunit H (ChIH; Sequence 24 (SEQ ID NO: 24)). It exhibits a reduction in chlorophyll in comparison to the WT strain UTEX 1230.

CS107 is characterised by mutations in the genes encoding magnesium-chelatase, subunit H (ChIH; Sequence 25 (SEQ ID NO: 25)) and geranylgeranyl diphosphate synthase (Sequence 26 (SEQ ID NO: 26)). It exhibits a reduction in chlorophyll in comparison to the WT strain UTEX 1230.

CS172 (whole genome sequence 64 (SEQ ID NO: 64)) is characterised by mutations in the genes encoding magnesium-chelatase, subunit H (ChIH; Sequence 25 (SEQ ID NO: 25)), geranylgeranyl diphosphate synthase (Sequence 26 (SEQ ID NO: 26)), and phytoene desaturase (Sequence 27, (SEQ ID NO: 27)). It exhibits a reduction in chlorophyll in comparison to the WT strain UTEX 1230.

CS174 is characterised by mutations in the genes encoding magnesium-chelatase, subunit H (ChIH; Sequence 25, (SEQ ID NO: 25)), geranylgeranyl diphosphate synthase (Sequence 26 (SEQ ID NO: 26)), and phytoene desaturase (Sequence 27 (SEQ ID NO: 27)). It exhibits a reduction in chlorophyll in comparison to the WT strain UTEX 1230.

CS10 is characterised by mutations in genes encoding magnesium chelatase, subunit H (ChIH; Sequence 24 (SEQ ID NO: 24)) and phytoene desaturase (EC: 1.3.5.5). It exhibits a reduction in chlorophyll in comparison to the WT strain UTEX 1230.

CS12 is characterised by mutations in the genes encoding magnesium-chelatase, subunit H (ChIH; Sequence 24 (SEQ ID NO: 24)) and phytoene desaturase (Sequence 28 (SEQ ID NO: 28), EC: 1.3.5.5). It exhibits a reduction in chlorophyll in comparison to the WT strain UTEX 1230.

CS120 is characterised by mutations in the genes encoding magnesium-chelatase, subunit I (ChII; Sequence 66 (SEQ ID NO: 66)) and phytoene desaturase (Sequence 65 (SEQ ID NO: 65), EC: 1.3.5.5). It exhibits a reduction in chlorophyll in comparison to the WT strain UTEX 1230.

### Production of Algae Biomass

To produce algae biomass, microalgal strains were cultivated at 100 L scale in a liquid fermentation medium.

The fermentation medium (FERM) described herein comprised: glucose (111 mM), (NH₄)₂SO₄ (47.7 mM), MgSO₄·7H₂O (2.8 mM), CaCl₂·2H₂O (204 μM), K₂HPO₄ (51.7 mM), NaH₂PO₄·H₂O (63.3 mM), KOH (40 mM), citric acid (8.8 mM), H₃BO₃ (1.1 mM), Na₂MoO₄ (32 μM), ZnSO₄·7H₂O (974 μM), MnSO₄·H₂O (958 M), NiCl₂·6H₂O (11 μM), FeSO₄·7H₂O (79.1 μM), CuSO₄·5H₂O (8 μM), Thiamine hydrochloride (5.65 M), Biotin (92.1 nM), Cyanocobalamin (13.3 nM), D-Pantothenic acid (205.3 nM), 4-Aminobenzoic acid (656.3 nM).

The high salt medium (HSM) described herein comprised: NH4Cl (7 mM), MgSO4·7H₂O (400 μM), CaCl2·2H2O (340 μM), K2HPO4 (4.13 mM), KH2PO4 (2.67 mM), Na2-EDTA (57.75 μM), (NH₄) 6Mo7O24·4H₂O (28.5 nM), Na2SeO3 (100 nM), ZnSO4·7H₂O (2.5 M), MnCl2·4H2O (6 μM), Na2CO3 (21.9 μM), FeCl3·6H2O (20 μM), CuCl2·2H2O (2 μM). Glucose is added at 1% (w/v) or 2% (w/v) or 3% (w/v) where indicated, in addition to: Thiamine hydrochloride (5.65 μM), Biotin (92.1 nM), Cyanocobalamin (13.3 nM), D-Pantothenic acid (205.3 nM), 4-Aminobenzoic acid (656.3 nM) to produce HSM1GV or HSM2GV or HSM3GV, respectively.

The microalgae biomass (e.g. flour) can be produced under current Good Manufacturing Practice (cGMP) conditions using any method known in the art. Typically, the procedure begins with the seed train phase, where a cryovial containing the microalgae culture is used to inoculate a series of increasing media volumes, culminating in a prepared inoculum for the fermentation phase. Fermentation is conducted in bioreactors with controlled environmental conditions.

Preferably, the microalgae biomass (e.g. flour) is produced by a three-step downstream process (DSP) from algae biomass, which is the product of a heterotrophic fermentation process described herein above. Following fermentation, the three-step DSP process the comprises the following steps: washing, concentration and drying. Packing is required at the end of the process. The purpose of the washing and concentration step is to reduce the spent medium carryover to a value that does not impact the organoleptic characteristics of the dried product and to achieve a dry cell weight concentration that favours the performance of the drying unit operation. Specifically, the biomass is washed once using an equivalent volume of city water and then concentrated using a nozzle centrifuge or a disk stack centrifuge with a self-discharging system to increase the biomass concentration up to 200 g/L. The biomass is then spray-dried using a stage spray drying system with external vibrating fluid bed in 15 s @ 80° C. Optionally, this process might not require washing at all. Further, optionally, concentrated biomass may be cracked, lysed or otherwise broken by mechanical means prior to drying.

Chlorella vulgaris Algae Biomass Production

In a first example of the production of algae biomass, Chlorella vulgaris microalgae strain WC03 was cultivated at 100 I scale in a liquid fermentation medium (FERM as described herein), beginning with a 1.5 mL cryovial that was used to inoculate a 50 mL flask, incubated for 7 days. The culture was then expanded in volume to 500 mL for 5 days, followed by a 5 L vessel for 5 to 6 days of either batch or fed-batch fermentation. The target initial density of biomass for the 100 L fermenter, referred to as the initial after inoculation concentration (AIC), was equal to or greater than 3 g/L. The final 100 L fermentation was closely controlled; pH and nitrogen requirements were managed by adding a 25% to 30% ammonia solution via the fermenter's pH control loop. The pH was maintained at 6.5. Glucose, the primary carbon source, was administered to maintain concentrations between 10 to 20 g/L, utilising either continuous feed or bolus additions to maintain this range. Temperature was maintained at 28° C., airflow rates were set between 0.5 to 1.0 vvm to facilitate oxygen transfer, with a fixed stirrer speed for consistent mixing. The fermentation process was completed with the separation of the biomass, followed by drying and packaging.

Chlorella sorokiniana Algae Biomass Production

In a second example of the production of algae biomass, Chlorella sorokiniana microalgae strain CS172 was cultivated at 100 I scale in a liquid fermentation medium (FERM as described herein), beginning with a 1.5 mL cryovial inoculating a 500 mL flask for 3 days. This was followed by a 2 to 3-day batch or fed-batch fermentation in a 5 L vessel. The mature culture was then inoculated in a 100 L fermenter for a 1.7 to 3-day fed-batch fermentation process, with a target AIC equal to or greater than 3 g/L. Glucose concentration in the fermentation broth was controlled within a 10 to 20 g/L range, and an ammonia solution at a 25% to 30% concentration was used to maintain pH at 6.5 and as a nitrogen feed. Temperature was maintained at 28° C., airflow rates were set between 0.5 to 1.0 vvm to facilitate oxygen transfer, with a fixed stirrer speed for consistent mixing. Following fermentation, the biomass underwent the downstream process of separation, drying, and packaging as described above, to obtain the final product.

Quantification of the Colour (CIELAB) of Algal Biomass (e.g. Flour) and Algae Biomass (e.g. Flour) Suspension

The colour of algal biomass (e.g. algal flour) and algal biomass (e.g. algal flour), resuspended in a specific volume of liquid at a specific solid %, can be quantified by a tristimulus colorimeter. The colorimeter works by quantifying the change in the intensity of electromagnetic radiation (within the visible wavelength spectrum 400 to 700 nm) after transmitting or reflectance; the absorbency of light waves, i.e the colour, can be measured. In a tristimulus, radiant power from a light source illuminates an object. The reflected or transmitted radiant power from the object channels is channelled through three independent tristimulus filters reaching a photo-detector. The response from the photodetector, is proportional to the corresponding tristimulus value of the object-source combination. This raw data is processed by a microprocessor for the computation of the absolute CIE tristimulus values. The values can be given as CIE LAB, XYZ, Lch, RGB and LUV.

In this instance the colour of Chlorella powder samples is measured using the PCE-CSM 2 colourimeter (https://www.pce-instruments.com/) according to the manufacturer's instructions. The device is calibrated only when first used, after significant environmental changes, after prolonged use or if results are inaccurate compared to a reference sample. To obtain the colour measurement, the powder compartment on the measuring plate is filled with Chlorella powder; overfilling without compacting, and the powder test box is assembled, ensuring a straight, snug screw joint and no air gaps under the glass in order to compact the powder and obtain an accurate measurement. The colorimeter is connected to a laptop using a USB cable and operated as per the manufacturer's instructions, using the supplied software which records CIEXYZ and CIELAB values. The colorimeter's measuring hole is placed over the test box and the measurement taken by pressing the test button. Three measurements are recorded per sample to obtain an average measurement. Between samples, the powder test box assembly is disassembled, wiped with a dry cloth and a fresh sample loaded as described above. Post-use, the test box assembly is cleaned with 70% (v/v) ethanol, dried and stored appropriately.

For liquid samples, a 5% solution is prepared by mixing 0.5 g of powder in 10 mL of deionised water. The mixture is allowed to hydrate at room temperature for 10 minutes, using a homogeniser for 5 seconds at maximum speed if necessary to produce a uniform, hydrated suspension. Immediately before the measurement is taken, the solution is remixed, before transferring to the powder compartment of the measuring plate using a Pasteur pipette; overfilling as before and removing any foam or bubbles by pipette aspiration. The test box is then assembled, avoiding bubbles and ensuring a straight, snug screw joint, and any overspill is dried to ensure it does not come into contact with the colourimeter. The colorimeter is operated as instructed by the manufacturer; following the same procedure as described above for powder samples to obtain triplicate measurements measurement. In between liquid samples, the test box is disassembled, rinsed with tap water and dried. After use, the test box is cleaned with 70% (v/v) ethanol, dried and stored appropriately.

A first comparison table showing the L*a*b* values of variants with a first mutation in at least one gene that encodes for phytoene desaturase or a subunit thereof, and i) a second mutation in at least one gene that encodes for subunit ChII of magnesium chelatase (Examples 1, 2 and 3—referred to above as WC03); and ii) a second mutation in at least one gene that encodes for subunit ChIH of magnesium chelatase magnesium chelatase or a subunit thereof (Examples 4, 5 and 6-referred to above as WC12, WCLS04 and WCLS06, respectively); is shown below in Table 9. Also included are the values for Comparative Example 1, which is a commercially available Chlorella-derived algae biomass. There is a statistically-significant difference in ΔE*ab values between strains with a magnesium chelatase subunit ChII and magnesium chelatase subunit ChIH mutation (two-tailed t.test, p<0.05, t=t=3.89, d.f=4).

Examples 1, 2, 3, 4, 5, and 6 were obtained by the methodology described above. Comparative Example 1 does not form part of the invention and was obtained commercially as “White Chlorella Powder” from www.allmashop.com.

TABLE 9
Colourimeter values of dried powder samples. ΔE*ab is the
measure of change in visual perception of two given colours,
in this case against pure white reference L* 100, a* 0, b* 0.

| | | CIELAB, 10° Observer, | | | | |
|---|---|---|---|---|---|---|
| | MgCl subunit | D 65 | | | | Mean |
| Sample | mutation | L* | a* | b* | ΔE*ab | ΔE*ab |
| Example 1 | ChII | 84.35 | 2.80 | 16.7133333 | 23.06 | 24.59 |
| Example 2 | ChII | 81.57 | 3.09 | 18.5233333 | 26.31 | |
| Example 3 | ChII | 83.45 | 1.84 | 17.8066667 | 24.38 | |
| Example 4 | ChIH | 80.81 | 2.93 | 20.46 | 28.20 | 29.7* |
| Example 5 | ChIH | 79.18 | 1.97 | 23.37 | 31.36 | |
| Example 6 | ChIH | 81.65 | 2.63 | 23 | 29.54 | |
| Comparative | Unknown | 77.23 | 4.05 | 26.79 | 35.39 | |
| Example 1 | | | | | | |

A two tail t-test was performed on the ΔE*ab values comparing samples with the mutation in magnesium chelatase subunit ChII (group 1) against samples with the mutation in magnesium chelatase subunit ChIH (group 2),
*indicates significant difference from ChII mean ΔE*ab value (p < 0.05, t = t = 3.89, d.f = 4).

A second comparison table showing the L*a*b* values of algae biomass according to the present invention is shown below in Table 10.

TABLE 10
Colourimeter values of dried powder samples.

| | | | Mean L* | Mean a* | Mean b* |
|---|---|---|---|---|---|
| Strain ID | Taxonomy | Colour | (StDev) | (StDev) | (StDev) |
| Comparative | | | 77.23 | 4.05 | 26.79 |
| example 1 | | | | | |
| WC03 | C. vulgaris | White | 85.500 (2.895) | 1.299 (0.898) | 15.740 (2.130) |
| WCLS06 | C. vulgaris | White | 83.650 (3.532) | 1.355 (0.614) | 17.243 (1.378) |
| CS172 | C. | White | 87.549 (1.661) | 0.766 (0.370) | 11.082 (1.208) |
| | sorokiniana | | | | |
| CS174 | C. | White | 87.790 (0.072) | 0.550 (0.010) | 11.937 (0.021) |
| | sorokiniana | | | | |

### Sequence Listings Index

Sequences 1-28 (SEQ ID NO: 1-28), 65 (SEQ ID NO: 65) and 66 (SEQ ID NO: 66) are individual genes.

Sequences 29-62 (SEQ ID NO: 29-62) are the whole genome sequence of C. vulgaris 4TC3, split into the contigs that are used to describe the position of variants.

Sequences 63 (SEQ ID NO: 63) and 64 (SEQ ID NO: 64) are the whole genome sequences of individual mutant strains.

| Sequence | | | If mutant which |
|---|---|---|---|
| number | Description | WT or mutant | strains |
| 1 (SEQ | Magnesium chelatase subunit | WT | |
| ID NO: 1) | ChII | | |
| 2 (SEQ | Magnesium chelatase subunit | Mutant | WC03 |
| ID NO: 2) | ChII | | |
| 3 (SEQ | Phytoene desaturase | WT | |
| ID NO: 3) | | | |
| 4 (SEQ | Phytoene desaturase | Mutant | WC03 |
| ID NO: 4) | | | |
| 5 (SEQ | Phytoene desaturase | Mutant | WC12 |
| ID NO: 5) | | | WCLS04 |
| | | | WCLS05 |
| | | | WCLS06 |
| 6 (SEQ | Magnesium chelatase subunit H | WT | |
| ID NO: 6) | | | |
| 7 (SEQ | Magnesium chelatase subunit H | Mutant | WC12 |
| ID NO: 7) | | | WCLS04 |
| | | | WCLS05 |
| | | | WCLS06 |
| 8 (SEQ | Alcohol dehydrogenase | WT | |
| ID NO: 8) | | | |
| 9 (SEQ | Alcohol dehydrogenase | Mutant | WC12 |
| ID NO: 9) | | | WCLS04 |
| | | | WCLS05 |
| | | | WCLS06 |
| 10 (SEQ | Starch binding domain | WT | |
| ID NO: | | | |
| 10) | | | |
| 11 (SEQ | Starch binding domain | Mutant | WC12 |
| ID NO: | | | WCLS04 |
| 11) | | | WCLS05 |
| | | | WCLS06 |
| 12 (SEQ | Glycogen phosphorylase | WT | |
| ID NO: | | | |
| 12) | | | |
| 13 (SEQ | Glycogen phosphorylase | Mutant | WCLS04 |
| ID NO: | | | |
| 13) | | | |
| 14 (SEQ | Cellulose synthase UDP forming | WT | |
| ID NO: | | | |
| 14) | | | |
| 15 (SEQ | Cellulose synthase UDP forming | Mutant | WCLS04 |
| ID NO: | | | |
| 15) | | | |
| 16 (SEQ | Isoamylase | WT | |
| ID NO: | | | |
| 16) | | | |
| 17 (SEQ | Isoamylase | Mutant | WCLS05 |
| ID NO: | | | |
| 17) | | | |
| 18 (SEQ | Glucose 6 phosphate isomerase | WT | |
| ID NO: | A | | |
| 18) | | | |
| 19 (SEQ | Glucose 6 phosphate isomerase | Mutant | WCLS05 |
| ID NO: | A | | |
| 19) | | | |
| 20 (SEQ | Glucose 6 phosphate isomerase | WT | |
| ID NO: | B | | |
| 20) | | | |
| 21 (SEQ | Glucose 6 phosphate isomerase | Mutant | WCLS06 |
| ID NO: | B | | |
| 21) | | | |
| 22 (SEQ | Trehalose 6 phosphate synthase | WT | |
| ID NO: | | | |
| 22) | | | |
| 23 (SEQ | Trehalose 6 phosphate synthase | Mutant | WCLS06 |
| ID NO: | | | |
| 23) | | | |
| 24 (SEQ | Magnesium Chelatase Subunit H | Mutant | CS04 |
| ID NO: | | | |
| 24) | | | |
| 25 (SEQ | Magnesium Chelatase Subunit H | Mutant | CS107 |
| ID NO: | | | CS172 |
| 25) | | | CS174 |
| 26 (SEQ | Geranylgeranyl diphosphate | Mutant | CS107 |
| ID NO: | synthase | | CS172 |
| 26) | | | CS174 |
| 27 (SEQ | Phytoene desaturase | Mutant | CS172 |
| ID NO: | | | CS174 |
| 27) | | | |
| 28 (SEQ | Phytoene desaturase | Mutant | CS12 |
| ID NO: | | | |
| 28) | | | |
| 29 (SEQ | Contig 1 | WT Whole | Chlorella vulgaris |
| ID NO: | | Genome Sequence | 4TC3 |
| 29) | | | |
| 30 (SEQ | Contig 2 | WT Whole | Chlorella vulgaris |
| ID NO: | | Genome Sequence | 4TC3 |
| 30) | | | |
| 31 (SEQ | Contig 3 | WT Whole | Chlorella vulgaris |
| ID NO: | | Genome Sequence | 4TC3 |
| 31) | | | |
| 32 (SEQ | Contig 4 | WT Whole | Chlorella vulgaris |
| ID NO: | | Genome Sequence | 4TC3 |
| 32) | | | |
| 33 (SEQ | Contig 5 | WT Whole | Chlorella vulgaris |
| ID NO: | | Genome Sequence | 4TC3 |
| 33) | | | |
| 34 (SEQ | Contig 6 | WT Whole | Chlorella vulgaris |
| ID NO: | | Genome Sequence | 4TC3 |
| 34) | | | |
| 35 (SEQ | Contig 7 | WT Whole | Chlorella vulgaris |
| ID NO: | | Genome Sequence | 4TC3 |
| 35) | | | |
| 36 (SEQ | Contig 8 | WT Whole | Chlorella vulgaris |
| ID NO: | | Genome Sequence | 4TC3 |
| 36) | | | |
| 37 (SEQ | Contig 9 | WT Whole | Chlorella vulgaris |
| ID NO: | | Genome Sequence | 4TC3 |
| 37) | | | |
| 38 (SEQ | Contig 10 | WT Whole | Chlorella vulgaris |
| ID NO: | | Genome Sequence | 4TC3 |
| 38) | | | |
| 39 (SEQ | Contig 11 | WT Whole | Chlorella vulgaris |
| ID NO: | | Genome Sequence | 4TC3 |
| 39) | | | |
| 40 (SEQ | Contig 12 | WT Whole | Chlorella vulgaris |
| ID NO: | | Genome Sequence | 4TC3 |
| 40) | | | |
| 41 (SEQ | Contig 13 | WT Whole | Chlorella vulgaris |
| ID NO: | | Genome Sequence | 4TC3 |
| 41) | | | |
| 42 (SEQ | Contig 14 | WT Whole | Chlorella vulgaris |
| ID NO: | | Genome Sequence | 4TC3 |
| 42) | | | |
| 43 (SEQ | Contig 15 | WT Whole | Chlorella vulgaris |
| ID NO: | | Genome Sequence | 4TC3 |
| 43) | | | |
| 44 (SEQ | Contig 16 | WT Whole | Chlorella vulgaris |
| ID NO: | | Genome Sequence | 4TC3 |
| 44) | | | |
| 45 (SEQ | Contig 17 | WT Whole | Chlorella vulgaris |
| ID NO: | | Genome Sequence | 4TC3 |
| 45) | | | |
| 46 (SEQ | Contig 18 | WT Whole | Chlorella vulgaris |
| ID NO: | | Genome Sequence | 4TC3 |
| 46) | | | |
| 47 (SEQ | Contig 19 | WT Whole | Chlorella vulgaris |
| ID NO: | | Genome Sequence | 4TC3 |
| 47 | | | |
| 48 (SEQ | Contig 20 | WT Whole | Chlorella vulgaris |
| ID NO: | | Genome Sequence | 4TC3 |
| 48) | | | |
| 49 (SEQ | Contig 21 | WT Whole | Chlorella vulgaris |
| ID NO: | | Genome Sequence | 4TC3 |
| 49) | | | |
| 50 (SEQ | Contig 22 | WT Whole | Chlorella vulgaris |
| ID NO: | | Genome Sequence | 4TC3 |
| 50) | | | |
| 51 (SEQ | Contig 23 | WT Whole | Chlorella vulgaris |
| ID NO: | | Genome Sequence | 4TC3 |
| 51) | | | |
| 52 (SEQ | Contig 24 | WT Whole | Chlorella vulgaris |
| ID NO: | | Genome Sequence | 4TC3 |
| 52) | | | |
| 53 (SEQ | Contig 25 | WT Whole | Chlorella vulgaris |
| ID NO: | | Genome Sequence | 4TC3 |
| 53) | | | |
| 54 (SEQ | Contig 26 | WT Whole | Chlorella vulgaris |
| ID NO: | | Genome Sequence | 4TC3 |
| 54) | | | |
| 55 (SEQ | Contig 27 | WT Whole | Chlorella vulgaris |
| ID NO: | | Genome Sequence | 4TC3 |
| 55) | | | |
| 56 (SEQ | Contig 28 | WT Whole | Chlorella vulgaris |
| ID NO: | | Genome Sequence | 4TC3 |
| 56) | | | |
| 57 (SEQ | Contig 29 | WT Whole | Chlorella vulgaris |
| ID NO: | | Genome Sequence | 4TC3 |
| 57) | | | |
| 58 (SEQ | Contig 30 | WT Whole | Chlorella vulgaris |
| ID NO: | | Genome Sequence | 4TC3 |
| 58) | | | |
| 59 (SEQ | Contig 31 | WT Whole | Chlorella vulgaris |
| ID NO: | | Genome Sequence | 4TC3 |
| 59) | | | |
| 60 (SEQ | Contig 32 | WT Whole | Chlorella vulgaris |
| ID NO: | | Genome Sequence | 4TC3 |
| 60) | | | |
| 61 (SEQ | Chloroplast | WT Whole | Chlorella vulgaris |
| ID NO: | | Genome Sequence | 4TC3 |
| 61) | | | |
| 62 (SEQ | Mitochondrion | WT Whole | Chlorella vulgaris |
| ID NO: | | Genome Sequence | 4TC3 |
| 62) | | | |
| 63 (SEQ | WCLS06 | Mutant Whole | WCLS06 |
| ID NO: | | Genome Sequence | |
| 63) | | | |
| 64 (SEQ | CS172 | Mutant Whole | CS172 |
| ID NO: | | Genome Sequence | |
| 64) | | | |
| 65 (SEQ | Phytoene desaturase | Mutant | CS120 |
| ID NO: | | | |
| 65) | | | |
| 66 (SEQ | Magnesium Chelatase Subunit I | Mutant | CS120 |
| ID NO: | | | |
| 66) | | | |

1. An algae biomass, wherein said biomass has a L* value in an L*a*b* colour space of greater than about 78.

2. The algae biomass of claim 1, having an a* value in an L*a*b* colour space in the range of between 0.1 and 4.0.

3. The algae biomass of claim 1 or claim 2, having a b* value in an L*a*b* colour space in the range of between 10 and 27.

4. The algae biomass of any preceding claim, wherein said biomass has a L* value in an L*a*b* colour space in the range of between 81.0 and 85.0; an a* value in the range of between 1.8 and 3.1; and a b* value in the range of between 16.0 and 19.0.

5. The algae biomass of any preceding claim, wherein said biomass has a protein content of at least 50% w/w.

6. The algae biomass of any preceding claim in the form of a powder or flour.

7. A Chlorella microalgae having a first mutation in at least one gene that encodes for phytoene desaturase or a subunit thereof, and wherein said Chlorella microalgae has a second mutation in at least one gene that encodes for magnesium chelatase or a subunit thereof.

8. The Chlorella microalgae of claim 7, wherein the second mutation is in a gene that encodes for subunit ChII of magnesium chelatase or subunit ChIH of magnesium chelatase.

9. The Chlorella microalgae of claim 8, wherein the second mutation is in a gene that encodes for subunit ChII of magnesium chelatase.

10. The Chlorella microalgae of any of claims 7 to 9, wherein the second mutation is a Single Nucleotide Polymorphism (SNP) or Insertion/deletion (INDEL).

11. The Chlorella microalgae of any of claims 7 to 10, being a modified strain of a Chlorella microalgae species.

12. The Chlorella microalgae of any of claims 7 to 11, wherein the Chlorella microalgae species is selected from Parachlorella kessleri, Auxenochlorella protothecoides, Auxenochlorella pyrenoidosa, or Heterochlorella luteoviridis, Chlorella sorokiniana or Chlorella vulgaris.

13. The Chlorella microalgae of any of claims 7 to 12, having a chlorophyll content in a range of 0.001-0.5 mg/g dry cell weight.

14. The Chlorella microalgae of any of claims 7 to 13, having a protein content of at least 50% w/w.

15. The Chlorella microalgae of any of claims 7 to 14, having a starch content of less than 25% w/w.

16. The Chlorella microalgae of any of claims 7 to 15, having a chitin and/or chitosan, poly-d-glucosamine and poly-acetyl-D-glucosamine content in a range of 0.001 to 4.8 mg/g dry cell weight.

17. The Chlorella microalgae of any of claims 7 to 16, having a protein digestibility-corrected amino acid score (PDCAAS) in a range of 0.75 to 1.

18. The Chlorella microalgae of any of claims 7 to 17, having one or more additional desirable phenotypes, wherein the one or more additional desirable phenotypes is selected from a group comprising: a smell, a taste, a texture, a biochemical composition and improved tolerance to process conditions.

19. The Chlorella microalgae of any of claims 7 to 18, obtained from a parent strain of Chlorella microalgae, by performing mutagenesis of the parent strain of Chlorella microalgae.

20. The Chlorella microalgae of claim 19, wherein mutagenesis is performed by exposure of the parent strain of Chlorella microalgae to a mutagenic chemical.

21. The Chlorella microalgae of claim 20, wherein the mutagenic chemical is an alkylating agent.

22. The Chlorella microalgae of claim 20 or claim 21, wherein the concentration of the mutagenic chemical is in a range from 0.1 to 2.0 M.

23. The Chlorella microalgae of claim 19, wherein mutagenesis is performed by exposure of the parent strain of Chlorella microalgae to a physical mutagen, optionally wherein the physical mutagen comprises at least one of UV light, gamma rays, X-rays.

24. The Chlorella microalgae of any preceding claim, cultivated in a heterotrophic growth mode.

25. The Chlorella microalgae of any preceding claim, cultivated:
at a specific temperature;
for a predefined period of time;
without the presence of light; and
in the presence of an organic carbon energy source.

26. The Chlorella microalgae of claim 25, wherein the specific temperature is in a range of 20 to 35° C., optionally in the range of 25 to 28° C.

27. The Chlorella microalgae of claim 25 or claim 26, wherein the predefined period of time is in a range of 1 to 5 weeks, optionally in the range of 1 to 3 weeks.

28. The Chlorella microalgae of any one of claims 25 to 27, wherein the organic carbon energy source is glucose and/or acetate.

29. The Chlorella microalgae of claim 28, wherein the organic carbon energy source is glucose having a glucose to biomass conversion ratio of more than 0.45.

30. The Chlorella microalgae of any of claims 7 to 29, being genetically stable.

31. A Chlorella microalgae comprising a genomic DNA sequence that is at least 50% identical to Sequence 2 (SEQ ID NO: 2).

32. An algae biomass derived from the Chlorella microalgae of any one of claims 7 to 31.

33. A protein isolate or concentrate derived from an algae biomass of claim 32, or derived from the Chlorella microalgae of any one of claims 7 to 31.

34. A composition comprising an algae biomass of any of claims 1 to 6 or claim 32, or a protein isolate or concentrate of claim 33, employed in at least one of: human foods, human nutraceutical preparations or formulations, animal feeds, pharmaceutical compositions including vaccines, cosmetics, personal care compositions, personal care devices.

35. A method of producing a Chlorella microalgae, the method comprising:
a) obtaining a parent strain of Chlorella microalgae;
b) performing mutagenesis of the parent strain of Chlorella microalgae;
c) cultivating the mutated strain of Chlorella microalgae at a specific temperature, for a predefined period of time, and in the presence of an organic carbon source; and
d) identifying and isolating mutants of the parent strain of Chlorella microalgae having a mutation in at least one gene that encodes for phytoene desaturase or a subunit thereof, and further having a mutation in at least one gene that encodes for magnesium chelatase or a subunit thereof.

36. A Chlorella microalgae strain selected from the following:
(i) a Chlorella vulgaris strain designated CCAP 211/143 deposited under provisions of the Budapest Treaty at the Culture Collection of Algae and Protozoa (CCAP, SAMS Ltd., Scottish Marine Institute, OBAN, Argyll, PA37 1QA, United Kingdom) on Dec. 14, 2023 (Patent Deposit Designation of CCAP 211/143);
(ii) a Chlorella sorokiniana strain designated CCAP 211/142 deposited under provisions of the Budapest Treaty at the Culture Collection of Algae and Protozoa (CCAP, SAMS Ltd., Scottish Marine Institute, OBAN, Argyll, PA37 1QA, United Kingdom) on Dec. 14, 2023 (Patent Deposit Designation of CCAP 211/142).
